(19) United States
(12) Patent Application Publication
(10) Pub. No.: US 2006/0134129 A1
(43) Pub. Date: Jun. 22, 2006

(54) Synthetic HLA binding peptide analogues and uses thereof

(76) Inventors: David A. Scheinberg, New York, NY (US); Javier Pinilla-Ibarz, New York, NY (US)

Correspondence Address: PEARL COHEN ZEDEK, LLP, 1500 BROADWAY 12TH FLOOR, NEW YORK, NY, 10036, US

(21) Appl. No.: 11/238,128

(22) Filed: Sep. 29, 2005

Related U.S. Application Data
(63) Continuation-in-part of application No. 10/999,425, filed on Nov. 30, 2004.
(60) Provisional application No. 60/525,955, filed on Dec. 1, 2003.

(51) Int. Cl. A61K 48/00; A61K 39/00; A61K 38/10

(52) U.S. Cl. 424/185.1; 514/15; 514/44

(57) ABSTRACT

The present invention is directed to immunogenic bcr-abl-based peptides, compositions and vaccines comprising same, and methods of use thereof for treating, inhibiting or reducing the incidence of a bcr-abl-expressing cancer, and methods of generating a heteroclitic immune response against, or cytotoxic T cells specifically recognizing bcr-abl-expressing cancer cells.

### CROSS REFERENCE TO RELATED APPLICATIONS

This application is a continuation-in-part of U.S. application Ser. No. 10/999,425, filed Nov. 30, 2004, which claims priority of U.S. Provisional Application Ser. No. 60/525,955, filed Dec. 1, 2003. These applications are hereby incorporated in their entirety by reference herein.

### STATEMENT REGARDING FEDERALLY SPONSORED RESEARCH OR DEVELOPMENT

This invention was produced in part using funds obtained through National Institutes of Health Grant Nos. PO1 23766 and RO1 55349. Consequently, the federal government has certain rights in this invention.

### FIELD OF INVENTION

The present invention is directed to immunogenic bcr-abl-based peptides, compositions and vaccines comprising same, and methods of use thereof for treating, inhibiting or reducing the incidence of a bcr-abl-expressing cancer, and methods of generating a heteroclitic immune response against, or cytotoxic T cells specifically recognizing bcr-abl-expressing cancer cells.

### BACKGROUND OF THE INVENTION

Leukemias, including chronic myelogenous leukemia (CML), acute myelogenous leukemia (AML) and acute lymphocytic leukemia (ALL) are pluripotent stem cell disorders, which may be characterized by the presence of the Philadelphia chromosome (Ph). Because of the unique features, these cancers present a unique opportunity to develop therapeutic strategies using vaccination against a truly tumor specific antigen that is also the oncogenic protein required for neoplasia.

The chimeric fusion proteins are potential antigens for two reasons. The proteins are uniquely expressed in the leukemic cells in which the junctional regions contain a sequence of amino acids that is not expressed on any normal protein. In addition, as a result of the codon split on the fused message, a new amino acid (lysine in b3a2) and a conserved one (glutamic acid in b2a2) are present at the exact fusion point in each of the proteins. Therefore, the unique amino acid sequences encompassing the b3a2 and b2a2 breakpoint region can be considered truly tumor specific antigens. Despite the intracellular location of these proteins, short peptides produced by cellular processing of the products of the fusion proteins can be presented on the cell surface within the cleft of HLA molecules, and in this form, may be recognized by T cells.

In previous experiments, tumor-specific vaccines containing peptides corresponding to the original bcr-abl sequence were safely administered to patients with chronic phase CML. While the vaccines elicited a bcr-abl peptide-specific CD4⁺ immune response, CD8⁺ responses were undetectable in HLA-A0201 patients (e.g. patients with the -0201 allele at HLA-A, an MHC class I molecule locus), and only weak responses were detected in HLA-A0301 patients.

The strength of CD8⁺ responses elicted by peptide vaccines responses depends upon the binding affinity of the target peptide to class I MHC molecules, the peptide-HLA complex stability, and the avidity of the T cell receptor binding for the peptide complex. Killing of target cells by CTL also requires adequate processing of the natural antigen and presentation of peptide(s) corresponding to the vaccine peptide(s). Therefore the lack elicitation of reproducible CD8⁺ responses by the previous bcr-abl vaccine likely reflects lack of affinity of the peptides for MHC class I molecules, which resulted in their weak immunogenicity to CTL.

Thus, there remains a need to design peptides that are more immunogenic and that produce a robust CTL response.

### SUMMARY OF THE INVENTION

The present invention is directed to immunogenic bcr-abl-based peptides, compositions and vaccines comprising same, and methods of use thereof for treating, inhibiting or reducing the incidence of a bcr-abl-expressing cancer, and methods of generating a heteroclitic immune response against, or cytotoxic T cells specifically recognizing bcr-abl-expressing cancer cells.

As provided herein, peptides derived from bcr-abl breakpoint regions were designed. In some of the peptides, single- or double AA substitutions were introduced at key HLA A0201 binding positions. The peptides were found to stimulate T lymphocytes that produced interferon-γ and lysed target cells.

In one embodiment, the present invention provides a bcr-abl-based peptide, comprising a sequence of amino acids that is an analogue peptide of a native bcr-abl breakpoint peptide that specifically binds to a HLA molecule on a cell surface. In another embodiment, the bcr-abl-based peptide specifically binds to the HLA molecule on a cell surface with a greater affinity than the native bcr-abl breakpoint peptide. In another embodiment, the peptide is heteroclitic.

In another embodiment, the present invention provides a method of inducing in a subject formation and proliferation of human cytotoxic T cells (CTL) that recognize a cancer cell, wherein the cancer cell presents the native bcr-abl breakpoint peptide of the present invention on a major histocompatibility complex (MHC) class I molecule thereof, the method comprising contacting the subject with a bcr-abl-based peptide of the present invention, whereby the bcr-abl-based peptide induces formation and proliferation of the human CTL.

In another embodiment, the present invention provides a method of treating a subject having a bcr-abl-expressing cancer, wherein a cell of the cancer presents the native bcr-abl breakpoint peptide of the present invention on a MHC class I molecule thereof, comprising (a) inducing in a donor formation and proliferation of human CTL that recognize the cell by the method of the present invention; (b) removing the human CTL from the donor; and (c) infusing the human CTL into the subject, thereby treating a subject having a bcr-abl-expressing cancer.

In another embodiment, the present invention provides a method of treating a subject having a bcr-abl-expressing cancer, wherein a cell of the cancer presents the native bcr-abl breakpoint peptide of the present invention on a MHC class I molecule thereof, comprising (a) removing human immune cells from a donor; (b) contacting ex vivo the human immune cells with the bcr-abl-based peptide of the present invention, whereby the bcr-abl-based peptide induces formation and proliferation of CTL that recognize the cell of the cancer; and (c) infusing the CTL into the subject, thereby treating a subject having a bcr-abl-expressing cancer.

In another embodiment, the present invention provides a method of inducing a heteroclitic immune response against a bcr-abl-expressing leukemia cell in a human, the method comprising administering to the human an effective amount of a pharmaceutical composition, the pharmaceutical composition comprising: (a) a therapeutically effective amount of a bcr-abl-based peptide of the present invention or a DNA encoding the bcr-abl-based peptide; and (b) a pharmaceutically acceptable carrier, whereby the leukemia cell presents the native bcr-abl breakpoint peptide of the present invention, thereby inducing formation and proliferation of CTL that recognize the leukemia cell. Thus, a heteroclitic immune response against a bcr-abl-expressing leukemia cell is induced in a human.

In another embodiment, the present invention provides a method for treating a leukemia in a subject, comprising inducing a heteroclitic immune response against a leukemia cell in a donor by the method of the present invention, and infusing the CTL of the present invention to the subject.

### BRIEF DESCRIPTION OF THE FIGURES

FIG. 1 depicts results of a T2 stabilization assay using peptides derived from b3a2 translocation (left panel) and b2a2 translocations (right panel). Peptide sequences are delineated in Table 1. The fluorescence index is the value obtained for the ratio between median fluorescence obtained with the indicated peptide divided by background fluorescence. The X-axis represents different peptide concentrations. “n” denotes native sequences from b3a2. p210Cn, p210Dn, CMLA2, and CMLA3 are native b3a2 sequences; b2a2A is the native sequence for b2a2.

FIG. 2 depicts gamma interferon (IFN) production detected by ELISPOT of CD8⁺ T cells from a healthy HLA A0201 donor following two in vitro stimulations with the peptides p210 C and F. After stimulation, CD8⁺ cells were challenged with the following: T2 (APC), or T2 pulsed with tested peptide (p210C or p210F), corresponding native peptide, or negative control peptide, as indicated.

FIG. 3 depicts secretion of gamma IFN detected by ELISPOT of CD8⁺ T cells from an HLA A0201, chronic phase CML patient following two in vitro stimulations with p210C. T cells were challenged with the following: media, APC T2, or T2 pulsed with p210C, corresponding native peptide, or negative control peptide. Empty bars: CD8+ cells plus media. Dot bars: CD8+ plus APC T2. Diagonal bars: CD8+ plus T2 pulsed with p210C. Black bars: CD8+ plus T2 pulsed with corresponding native peptide p210Cn. Grey bars: CD8+ plus T2 pulsed with irrelevant control peptide.

FIG. 4 depicts production of gamma IFN detected by ELISPOT of CD3⁺ cells of two healthy HLA A0201 donors after two in vitro stimulations. T cells were challenged with the following: media, APC T2, or T2 pulsed with test peptide (b2a2 A3, A4 or A5); corresponding native peptide, or negative control peptide. Dot bars: CD8+ plus APC T2. diagonal bars: CD8+ plus T2 pulsed with tested peptide (b2a2 A3, A4 or A5). black bars: CD8+ plus T2 pulsed with native peptide (cross reactivity). grey bars: CD8+ plus T2 pulsed with irrelevant control peptide.

FIG. 5 depicts results of a cytotoxicity assay with T cells isolated from a healthy HLA A0201 donor following three in vitro stimulations with p210F. Target cells used were T2 cell lines pulsed with the indicated peptides. The Y-axis reflects the percent cytotoxicity, and the X-axis reflects the varied T cell/target ratio. Open squares: T2 with no peptide. Open diamonds: T2 pulsed with p210F. Open circles: T2 pulsed with CMLA2. Open triangles: T2 pulsed with irrelevant control peptide.

FIG. 6 depicts results of two cytotoxicity assays with T cells isolated from a healthy HLA A0201 donor following five in vitro stimulations with b2a2 A3 peptide. Target cells used were T2 cell line pulsed with the indicated peptides. Y-axis reflects the percent cytotoxicity, and the X-axis reflects the different T cell/target ratio. Open squares: T2 with no peptide. Open diamond: T2 pulsed with b2a2 A3 peptide. Open circles: T2 pulsed with negative control peptide.

### DETAILED DESCRIPTION OF THE INVENTION

The present invention is directed to immunogenic bcr-abl-based peptides, compositions and vaccines comprising same, and methods of use thereof for treating, inhibiting or reducing the incidence of a bcr-abl-expressing cancer, and methods of generating a heteroclitic immune response against, or cytotoxic T cells specifically recognizing bcr-abl-expressing cancer cells.

As provided herein, peptides derived from bcr-abl breakpoint regions were designed. In some of the peptides, single- or double AA substitutions were introduced at key HLA A0201 binding positions. The peptides were found to stimulate T lymphocytes that produced interferon-γ and lysed target cells.

In one embodiment, the present invention provides a bcr-abl-based peptide, comprising a sequence of amino acids that is an analogue peptide of a native bcr-abl breakpoint peptide that specifically binds to a HLA molecule on a cell surface. In another embodiment, the bcr-abl-based peptide specifically binds to the HLA molecule on a cell surface with a greater affinity than the native bcr-abl breakpoint peptide. In another embodiment, the peptide is heteroclitic.

Bcr-abl is, in one embodiment, a fusion gene associated, inter alia, with chronic myelogenous leukemia (CML), and results from a translocation of the c-abl oncogene from chromosome 9 to the specific breakpoint cluster region (bcr) of the BCR gene on chromosome 22. The t(9;22) (q34; q11) translocation is present in more than 95% of patients with CML. The translocation of the c-abl to the breakpoint cluster region (bcr) forms bcr-abl, which, in one embodiment, is a 210 kD chimeric protein with abnormal tyrosine kinase activity.

In another embodiment, bcr-abl is typically expressed only by leukemia cells. In another embodiment, bcr-abl can stimulate the growth of hematopoietic progenitor cells and contributes to pathogenesis of leukemia, which, in another embodiment, is CML. In other embodiments, the bcr breakpoint is between exons 2 and 3 or exons 3 and 4. In another embodiment, the bcr-abl reading frames are fused in frame, and the translocated mRNA encodes a functional 210 kD chimeric protein consisting of 1,004 c-abl encoded amino acids plus either 902 or 927 bcr encoded amino acids—both of which are enzymatically active as protein kinases. Each bcr-abl protein represents a separate embodiment of the present invention.

In one embodiment, the native bcr-abl breakpoint peptide is a p210-b3a2 peptide. In another embodiment, the native bcr-abl breakpoint peptide is a p210-b2a2 peptide. In another embodiment, the native bcr-abl breakpoint peptide is a p190-ela2 peptide. In another embodiment, the native bcr-abl breakpoint peptide is from any other bcr-abl protein known in the art. Each possibility represents a separate embodiment of the present invention.

In another embodiment, bcr-abl peptides of methods and compositions of the present invention are derived from junctional sequences. “Junctional sequences” (“breakpoint sequences”) refers, in one embodiment, to sequences that span the fusion point of bcr-abl or another protein that arises from a translocation.

The bcr-abl protein of methods and compositions of the present invention can be any bcr-abl protein known in the art. In another embodiment, the bcr-abl protein has the sequence set forth in GenBank Accession # M14752. In other embodiments, the bcr-abl protein has one of the following sequences: BAB62851, AAL05889, AAL99544, CAA10377, CAA10376, AAD04633, AAF89176, AAA35596, AAF61858, 1314255A, AAA88013, AAA87612, AAA35594, and AAA35592. In another embodiment, the bcr-protein has any other bcr-abl sequence known in the art.

In another embodiment, the bcr-abl protein is derived from the translated product of a bcr-abl translocation event that is associated with a neoplasm. In one embodiment, the neoplasm is a leukemia, which is, in other embodiments, a chronic or acute myelogenous or acute lymphoblastic leukemia.

In another embodiment, the bcr-abl protein of methods and compositions of the present invention results from a translocation associated with acute lymphoblastic leukemia (ALL), wherein c-abl is translocated to chromosome 22 but to a different region of the bcr gene, denoted BCRI, which results in the expression of a p185-190^{bcr-abl} chimeric protein kinase. p185-190^{bcr-abl} is expressed in approximately 10% of children and 25% of adults with ALL.

Each of the above bcr-abl proteins or types thereof represents a separate embodiment of the present invention.

“Peptide,” in one embodiment of methods and compositions of the present invention, refers to a compound of two or more subunit AA connected by peptide bonds. In another embodiment, the peptide comprises an AA analogue. In another embodiment, the peptide comprises a peptidomimetic. The different AA analogues and peptidomimetics that can be included in the peptides of methods and compositions of the present invention are enumerated hereinbelow. The subunits are, in another embodiment, linked by peptide bonds. In another embodiment, the subunit is linked by another type of bond, e.g. ester, ether, etc. Each possibility represents a separate embodiment of the present invention.

In one embodiment, a peptide of the present invention is immunogenic. In one embodiment, the term “immunogenic” refers to an ability to stimulate, elicit or participate in an immune response. In one embodiment, the immune response elicited is a cell-mediated immune response. In another embodiment, the immune response is a combination of cell-mediated and humoral responses.

In another embodiment, the peptide of methods and compositions of the present invention is so designed as to exhibit affinity for a major histocompatibility complex (MHC) molecule. In one embodiment, the affinity is a high affinity, as described herein.

In another embodiment, T cells that bind to the MHC molecule-peptide complex become activated and induced to proliferate and lyse cells expressing a protein comprising the peptide. T cells are typically initially activated by “professional” antigen presenting cells (e.g. dendritic cells, monocytes, and macrophages), which present costimulatory molecules that encourage T cell activation as opposed to anergy or apoptosis. In another embodiment, the response is heteroclitic, as described herein, such that the CTL lyses a neoplastic cell expressing a protein which has an AA sequence homologous to a peptide of this invention, or a different peptide than that used to first stimulate the T cell.

In another embodiment, an encounter of a T cell with a peptide of this invention induces its differentiation into an effector and/or memory T cell. Subsequent encounters between the effector or memory T cell and the same peptide, or, in another embodiment, with a related peptide of this invention, leads to a faster and more intense immune response. Such responses are gauged, in one embodiment, by measuring the degree of proliferation of the T cell population exposed to the peptide. In another embodiment, such responses are gauged by any of the methods enumerated hereinbelow.

In another embodiment, the peptides of methods and compositions of the present invention bind an HLA class I molecule with high affinity. In another embodiment, the peptides bind an HLA class II molecule with high affinity. In another embodiment, the peptides bind both an HLA class I molecule and an HLA class II molecule with good In other embodiment, the MHC class I molecule is encoded by any of the HLA-A genes. In other embodiment, the MHC class I molecule is encoded by any of the HLA-B genes. In other embodiment, the MHC class I molecule is encoded by any of the HLA-C genes. In another embodiment, the MHC class I molecule is an HLA-0201 molecule. In another embodiment, the molecule is HLA A1. In other embodiments, the molecule is HLA A3.2, HLA A11, HLA A24, HLA B7, HLA B8, or HLA B27. In other embodiment, the MHC class II molecule is encoded by any of the HLA genes HLA-DP, -DQ, or -DR. Each possibility represents a separate embodiment of the present invention.

In one embodiment, “affinity” refers to the concentration of peptide necessary for inhibiting binding of a standard peptide to the indicated MHC molecule by fifty percent. In one embodiment, “high affinity” refers to an affinity is such that a concentration of about 500 nanomolar (nM) or less of the peptide is required for inhibition of binding of a standard peptide. In another embodiment, a concentration of about 400 nM or less of the peptide is required. In another embodiment, the binding affinity is 300 nM. In another embodiment, the binding affinity is 200 nM. In another embodiment, the binding affinity is 150 nM. In another embodiment, the binding affinity is 100 nM. In another embodiment, the binding affinity is 80 nM. In another embodiment, the binding affinity is 60 nM. In another embodiment, the binding affinity is 40 nM. In another embodiment, the binding affinity is 30 nM. In another embodiment, the binding affinity is 20 nM. In another embodiment, the binding affinity is 15 nM. In another embodiment, the binding affinity is 10 nM. In another embodiment, the binding affinity is 8 nM. In another embodiment, the binding affinity is 6 nM. In another embodiment, the binding affinity is 4 nM. In another embodiment, the binding affinity is 3 nM. In another embodiment, the binding affinity is 2 nM. In another embodiment, the binding affinity is 1.5 nM. In another embodiment, the binding affinity is 1 nM. In another embodiment, the binding affinity is 0.8 nM. In another embodiment, the binding affinity is 0.6 nM. In another embodiment, the binding affinity is 0.5 nM. In another embodiment, the binding affinity is 0.4 nM. In another embodiment, the binding affinity is 0.3 nM. In another embodiment, the binding affinity is less than 0.3 nM.

In another embodiment, “high affinity” refers to a binding affinity of 0.5-500 nM. In another embodiment, the binding affinity is 1-300 nM. In another embodiment, the binding affinity is 1.5-200 nM. In another embodiment, the binding affinity is 2-100 nM. In another embodiment, the binding affinity is 3-100 nM. In another embodiment, the binding affinity is 4-100 nM. In another embodiment, the binding affinity is 6-100 nM. In another embodiment, the binding affinity is 10-100 nM. In another embodiment, the binding affinity is 30-100 nM. In another embodiment, the binding affinity is 3-80 nM. In another embodiment, the binding affinity is 4-60 nM. In another embodiment, the binding affinity is 5-50 nM. In another embodiment, the binding affinity is 6-50 nM. In another embodiment, the binding affinity is 8-50 nM. In another embodiment, the binding affinity is 10-50 nM. In another embodiment, the binding affinity is 20-50 nM. In another embodiment, the binding affinity is 6-40 nM. In another embodiment, the binding affinity is 8-30 nM. In another embodiment, the binding affinity is 10-25 nM. In another embodiment, the binding affinity is 15-25 nM. Each affinity and range of affinities represents a separate embodiment of the present invention.

In another embodiment, the peptides of methods and compositions of the present invention bind to a superfamily of HLA molecules. Superfamilies of HLA molecules share very similar or identical binding motifs. (del Guercio M F, Sidney J, et al, 1995, J Immunol 154: 685-93; Fikes J D, and Sette A, Expert Opin Biol Ther. 2003 September; 3(6):985-93). In one embodiment, the superfamily is the A2 superfamily. In another embodiment, the superfamily is the A3 superfamily. In another embodiment, the superfamily is the A24 superfamily. In another embodiment, the superfamily is the B7 superfamily. In another embodiment, the superfamily is the B27 superfamily. In another embodiment, the superfamily is the B44 superfamily. In another embodiment, the superfamily is the C1 superfamily. In another embodiment, the superfamily is the C4 superfamily. In another embodiment, the superfamily is any other superfamily known in the art. Each possibility represents a separate embodiment of the present invention.

As provided herein, bcr-abl-derived peptides with high affinity to HLA-A0201 were identified. Immunogenicity of some of the peptides was improved by modifying anchor residues. The methods disclosed herein will be understood by those in the art to enable design of other bcr-abl breakpoint peptides, including heteroclitic peptides. The methods further enable design of peptides binding to HLA molecules other than HLA-A0201.

Each of the above peptides and types of peptides represents a separate embodiment of the present invention.

The analogue peptide contained in the bcr-abl-based peptide is, in another embodiment a degradation product thereof. In another embodiment, the analogue peptide binds an HLA molecule as part of a longer peptide; e.g. together with surrounding sequence from the bcr-abl-based peptide. In another embodiment, the analogue peptide is equivalent to the bcr-abl-based peptide. Each possibility represents a separate embodiment of the present invention.

In another embodiment, abcr-abl-based peptide of the present invention is extended relative to the native bcr-abl breakpoint peptide by adding additional surrounding sequence from the bcr-abl protein. In another embodiment, additional residues not from the bcr-abl surrounding sequence are added to the end of the bcr-abl-based peptide. In another embodiment, additional residues are added that enhance HLA binding. Each possibility represents a separate embodiment of the present invention.

In other embodiments, the analogue peptide has an amino acid sequence selected from the group consisting of YLKALQRPV (SEQ ID NO: 2), KQSSKALQV (SEQ ID NO: 4), KLSSKALQV (SEQ ID NO: 5), KLLQRPVAV (SEQ ID NO: 7), TLFKQSSKV (SEQ ID NO: 9), YLFKQSSKV (SEQ ID NO: 10), LLINKEEAL (SEQ ID NO: 12), LTINKVEAL (SEQ ID NO: 13), YLINKEEAL (SEQ ID NO: 14), YLINKEEAV (SEQ ID NO: 15), and YLINKVEAL (SEQ ID NO: 16). In other embodiments, the analogue peptide is another heteroclitic variant of one of the native bcr-abl breakpoint peptides listed herein. In other embodiments, the analogue peptide is another heteroclitic variant of any other native bcr-abl breakpoint peptides. In other embodiments, the analogue peptide is a variant or homologue of one of the sequences listed herein. Each possibility represents a separate embodiment of the present invention.

In other embodiments, the analogue peptide is a heteroclitic variant of one of the following native bcr-abl breakpoint peptides: SSKALQRPV (SEQ ID NO: 1), KQSSKALQR (SEQ ID NO: 3), KALQRPVAS (SEQ ID NO: 6), TGFKQSSKA (SEQ ID NO: 8), or LTINKEEAL (SEQ ID NO: 11). Each possibility represents a separate embodiment of the present invention.

In another embodiment, the analogue peptide contained in bcr-abl-based peptides of methods and compositions of the present invention differs from the native bcr-abl breakpoint peptide in a major histocompatibility complex (MHC) molecule anchor residue.

“Heteroclitic” refers, in one embodiment, to a peptide that generates an immune response that recognizes the original peptide from which the heteroclitic peptide was derived (e.g. the peptide not containing the anchor residue mutations). For example, KQSSKALQV is a heteroclitic peptide derived from KQSSKALQR, bcr-abl breakpoint native peptide, by mutation of the C-terminal residue to valine (Examples). In another embodiment, “heteroclitic” refers to a peptide that generates an immune response that recognizes the original peptide from which the heteroclitic peptide was derived, wherein the immune response generated by vaccination with the heteroclitic peptide is greater than the immune response generated by vaccination with the original peptide. In another embodiment, a “heteroclitic” immune response refers to an immune response that recognizes the original peptide from which the improved peptide was derived (e.g. the peptide not containing the anchor residue mutations). In another embodiment, a “heteroclitic” immune response refers to an immune response that recognizes the original peptide from which the heteroclitic peptide was derived, wherein the immune response generated by vaccination with the heteroclitic peptide is greater than the immune response generated by vaccination with the original peptide. Each possibility represents a separate embodiment of the present invention.

In one embodiment, the immune response induced by the peptides of this invention results in an increase of at least about 2-fold, or in another embodiment, 3-fold, or in another embodiment, 5-fold, or in another embodiment, 7-fold, or in another embodiment, 10-fold, or in another embodiment, 20-fold, or in another embodiment, 30-fold, or in another embodiment, 50-fold, or in another embodiment, 100-fold, or in another embodiment, 200-fold, or in another embodiment, 500-fold, or in another embodiment, 1000-fold, or in another embodiment, more than 1000-fold. Each possibility represents a separate embodiment of the present invention.

In another embodiment, the additional mutation that enhances MHC binding is in the residue at position 1 of the heteroclitic peptide. In one embodiment, the residue is changed to tyrosine. In another embodiment, the residue is changed to glycine. In another embodiment, the residue is changed to threonine. In another embodiment, the residue is changed to phenylalanine. In another embodiment, the residue is changed to any other residue known in the art. Each possibility represents a separate embodiment of the present invention.

In another embodiment, the additional mutation is in position 2 of the heteroclitic peptide. In one embodiment, the residue is changed to leucine. In another embodiment, the residue is changed to valine. In another embodiment, the residue is changed to isoleucine. In another embodiment, the residue is changed to methionine. In another embodiment, the residue is changed to any other residue known in the art. Each possibility represents a separate embodiment of the present invention.

In another embodiment, the additional mutation is in position 6 of the heteroclitic peptide. In one embodiment, the residue is changed to valine. In another embodiment, the residue is changed to cysteine. In another embodiment, the residue is changed to glutamine. In another embodiment, the residue is changed to histidine. In another embodiment, the residue is changed to any other residue known in the art. Each possibility represents a separate embodiment of the present invention.

In another embodiment, the additional mutation is in position 9 of the heteroclitic peptide. In another embodiment, the additional mutation changes the residue at the C-terminal position thereof. In one embodiment, the residue is changed to valine. In another embodiment, the residue is changed to threonine. In another embodiment, the residue is changed to isoleucine. In another embodiment, the residue is changed to leucine. In another embodiment, the residue is changed to alanine. In another embodiment, the residue is changed to cysteine. In another embodiment, the residue is changed to any other residue known in the art. Each possibility represents a separate embodiment of the present invention.

In other embodiments, the additional mutation is in the 3 position, the 4 position, the 5 position, the 7 position, or the 8 position. Each possibility represents a separate embodiment of the present invention.

The HLA molecule to which the bcr-abl-based peptide binds is, in one embodiment, an HLA A02 molecule (e.g. HLA A0201). In another embodiment, the HLA molecule is an HLA A03 molecule (e.g. HLA A0301). In other embodiment, the MHC class I molecule is encoded by any of the HLA-A genes. In other embodiment, the MHC class I molecule is encoded by any of the HLA-B genes. In other embodiment, the MHC class I molecule is encoded by any of the HLA-C genes. In another embodiment, the molecule is HLA A1. In other embodiments, the molecule is HLA A3.2, HLA A11, HLA A24, HLA B7, HLA B8, or HLA B27. In other embodiment, the MHC class I molecule is encoded by any of the HLA genes HLA-DP, -DQ, or -DR. Each possibility represents a separate embodiment of the present invention.

Bcr-abl-based peptides of the present invention can be of different lengths. In one embodiment, the length of the bcr-abl-based peptide is the same length as the native bcr-abl breakpoint peptide that is related to the analogue peptide contained in the bcr-abl-based peptide. In another embodiment, the length of the bcr-abl-based peptide is one amino acid (AA) shorter than the native bcr-abl breakpoint peptide. In another embodiment, the bcr-abl-based peptide is 2 AA shorter than the native bcr-abl breakpoint peptide. In another embodiment, the bcr-abl-based peptide is 3 AA shorter than the native bcr-abl breakpoint peptide. In another embodiment, the bcr-abl-based peptide is 4 AA shorter than the native bcr-abl breakpoint peptide. In another embodiment, the bcr-abl-based peptide is 5 AA shorter than the native bcr-abl breakpoint peptide. In another embodiment, the bcr-abl-based peptide is more than 5 AA shorter than the native bcr-abl breakpoint peptide. In another embodiment, the bcr-abl-based peptide is 1 AA longer than the native bcr-abl breakpoint peptide. In another embodiment, the bcr-abl-based peptide is 2 AA longer than the native bcr-abl breakpoint peptide. In another embodiment, the bcr-abl-based peptide is 3 AA longer than the native bcr-abl breakpoint peptide. In another embodiment, the bcr-abl-based peptide is 4 AA longer than the native bcr-abl breakpoint peptide. In another embodiment, the bcr-abl-based peptide is 5 AA longer than the native bcr-abl breakpoint peptide. In another embodiment, the bcr-abl-based peptide is more than 5 AA longer than the native bcr-abl breakpoint peptide.

In another embodiment, the length of the bcr-abl-based peptide is about 5% shorter than the native bcr-abl breakpoint peptide. In another embodiment, the bcr-abl-based peptide is about 5% shorter than the native bcr-abl breakpoint peptide. In another embodiment, the bcr-abl-based peptide is about 10% shorter than the native bcr-abl breakpoint peptide. In another embodiment, the bcr-abl-based peptide is about 15% shorter than the native bcr-abl breakpoint peptide. In another embodiment, the bcr-abl-based peptide is about 20% shorter than the native bcr-abl breakpoint peptide. In another embodiment, the bcr-abl-based peptide is about 30% shorter than the native bcr-abl breakpoint peptide. In another embodiment, the bcr-abl-based peptide is more than 30% shorter than the native bcr-abl breakpoint peptide. In another embodiment, the bcr-abl-based peptide is about 5% longer than the native bcr-abl breakpoint peptide. In another embodiment, the bcr-abl-based peptide is about 10% longer than the native bcr-abl breakpoint peptide. In another embodiment, the bcr-abl-based peptide is about 15% longer than the native bcr-abl breakpoint peptide. In another embodiment, the bcr-abl-based peptide is about 20% longer than the native bcr-abl breakpoint peptide. In another embodiment, the bcr-abl-based peptide is about 25% longer than the native bcr-abl breakpoint peptide. In another embodiment, the bcr-abl-based peptide is about 30% longer than the native bcr-abl breakpoint peptide. In another embodiment, the bcr-abl-based peptide is more than 30% longer than the native bcr-abl breakpoint peptide.

In another embodiment, the length of the bcr-abl-based peptide differs by 5-10% from the native bcr-abl breakpoint peptide. In another embodiment, the length differs by 5-15%. In another embodiment, the length differs by 10-20%. In another embodiment, the length differs by 15-25%. In another embodiment, the length differs by 20-30%. In another embodiment, the length differs by 5-20%. In another embodiment, the length differs by 5-30%. In another embodiment, the length differs by 10-25%. In another embodiment, the length differs by 10-30%. In another embodiment, the length differs by 10-40%. In another embodiment, the length differs by 10-50%. In another embodiment, the length differs by 30-40%. In another embodiment, the length differs by 40-50%. In another embodiment, the length differs by 50-60%. In another embodiment, the length differs by 50-75%. In another embodiment, the length differs by 50-100%.

In another embodiment, a bcr-abl peptide of the present invention has a length of 8 AA. In another embodiment, the length is 9 AA. In another embodiment, the length is 10 AA. In another embodiment, the length is 11 AA. In another embodiment, the length is 12 AA. In another embodiment, the length is 13 AA. In another embodiment, the length is 14 AA. In another embodiment, the length is 15 AA. In another embodiment, the length is more than 15 AA. In another embodiment, the length is about 20 AA. In another embodiment, the length is about 25 AA. In another embodiment, the length is about 30 AA. In another embodiment, the length is 8-12 AA. In another embodiment, the length is 9-12 AA. In another embodiment, the length is 8-11 AA. In another embodiment, the length is 8-10 AA. In another embodiment, the length is 8-9 AA. In another embodiment, the length is 9-11 AA. In another embodiment, the length is 9-10 AA. In another embodiment, the length is 9-13 AA. In another embodiment, the length is 9-14 AA. In another embodiment, the length is 9-15 AA. In another embodiment, the length is 9-16 AA.

Each length represents a separate embodiment of the present invention.

In another embodiment, the present invention provides a method of inducing in a subject formation and proliferation of human cytotoxic T cells (CTL) that recognize a cancer cell, wherein the cancer cell presents the native bcr-abl breakpoint peptide of the present invention on a major histocompatibility complex (MHC) class I molecule thereof, the method comprising contacting the subject with a bcr-abl-based peptide of the present invention, whereby the bcr-abl-based peptide induces formation and proliferation of the human CTL.

In another embodiment, the present invention provides a method of treating a subject having a bcr-abl-expressing cancer, wherein a cell of the cancer presents the native bcr-abl breakpoint peptide of the present invention on a MHC class I molecule thereof, comprising (a) inducing in a donor formation and proliferation of human CTL that recognize the cell by the method of the present invention; (b) removing the human CTL from the donor; and (c) infusing the human CTL into the subject, thereby treating a subject having a bcr-abl-expressing cancer.

“Removing” the CTL from the donor refers, in one embodiment, to removing an effective amount of the CTL. In another embodiment, the term refers to removing a pre-determined amount from the donor. In another embodiment, the term refers to removing as many cell as are obtained by the technique used. Each possibility represents a separate embodiment of the present invention.

In another embodiment, the present invention provides a method of treating a subject having a bcr-abl-expressing cancer, wherein a cell of the cancer presents the native bcr-abl breakpoint peptide of the present invention on a MHC class I molecule thereof, comprising (a) removing human immune cells from a donor; (b) contacting ex vivo the human immune cells with the bcr-abl-based peptide of the present invention, whereby the bcr-abl-based peptide induces formation and proliferation of CTL that recognize the cell of the cancer; and (c) infusing the CTL into the subject, thereby treating a subject having a bcr-abl-expressing cancer.

In another embodiment, the present invention provides a method of inducing a heteroclitic immune response against a bcr-abl-expressing leukemia cell in a human, the method comprising administering to the human an effective amount of a pharmaceutical composition, the pharmaceutical composition comprising: (a) a therapeutically effective amount of a bcr-abl-based peptide of the present invention or a DNA encoding the bcr-abl-based peptide; and (b) a pharmaceutically acceptable carrier, whereby the leukemia cell presents the native bcr-abl breakpoint peptide of the present invention, thereby inducing formation and proliferation of CTL that recognize the leukemia cell. Thus, a heteroclitic immune response against a bcr-abl-expressing leukemia cell is induced in a human.

In another embodiment, the present invention provides a method for treating a leukemia in a subject, comprising inducing a heteroclitic immune response against a leukemia cell in a donor by the method of the present invention, and infusing the CTL of the present invention to the subject.

In another embodiment, the bcr-abl-based peptide utilized in methods of the present invention is produced by contacting the subject with a DNA molecule encoding the bcr-abl-based peptide. In another embodiment, the bcr-abl-based peptide is produced in situ by any other method known in the art. Each possibility represents a separate embodiment of the present invention.

In another embodiment, multiple peptides of this invention are used to stimulate an immune response in methods of the present invention.

In one embodiment, the subject of methods of the present invention is a human. In one embodiment, the human has an active cancer. In another embodiment, the human is in remission from cancer. In another embodiment, the human is at risk of developing a cancer. Each possibility represents a separate embodiment of the present invention.

In one embodiment, the leukemia treated by a method of the present invention is a CML. In another embodiment, the leukemia is an AML. In another embodiment, the leukemia is an ALL. In another embodiment, the leukemia is any other type of leukemia known in the art. Each possibility represents a separate embodiment of the present invention.

In another embodiment, methods of the present invention utilize human immune cells. In one embodiment, the human immune cells are peripheral blood mononuclear cells. In another embodiment, the human immune cells are bone marrow cells. In another embodiment, the human immune cells are dendritic cells. In another embodiment, the human immune cells are macrophages. Each possibility represents a separate embodiment of the present invention.

In another embodiment of methods of the present invention, the human immune cells are contacted in vivo in a donor by a peptide, composition, or vaccine of the present invention. The resulting CTL that are generated are removed from the donor; and infused into an individual having a cancer.

The cancer that is treated by methods of the present invention is, in one embodiment, a leukemia. In another embodiment, the cancer is chronic myelogenous leukemia (CML). In another embodiment, the cancer is a breast cancer. In another embodiment, the cancer is a lymphoma. In another embodiment, the cancer is a mesothelioma. In another embodiment, the cancer is a lung cancer. In another embodiment, the cancer is a testicular cancer. In another embodiment, the cancer is an ovarian cancer. In another embodiment, the cancer is any other type of cancer known in the art. In a similar vein, the cancer cells against which an immune response is generated by methods of the present invention can be, in other embodiments, cells of any of the above types of cancer. Each possibility represents a separate embodiment of the present invention.

In another embodiment, the terms “cancer,” “neoplasm,” “neoplastic” or “tumor,” may be used interchangeably and refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. “Cancer cell,” in another embodiment, includes not only a primary cancer cell, but also any cell derived from a cancer cell ancestor. This includes metastasized cancer cells, and in vitro cultures and cell lines derived from cancer cells. In one embodiment, a tumor is detectable on the basis of tumor mass; e.g., by such procedures as CAT scan, magnetic resonance imaging (MRI), X-ray, ultrasound or palpation, and in another embodiment, is identified by biochemical or immunologic findings, the latter which is used to identify cancerous cells, as well, in other embodiments.

In other embodiments, any of the above types of cancer are treated by methods of the present invention. Each type of cancer or its treatment by a method of the present invention represents a separate embodiment of the present invention.

In other embodiments, the peptides utilized in any of the methods described above have any of the characteristics of a peptide of the present invention. Each characteristic represents a separate embodiment of the present invention.

In one embodiment, a treatment protocol of the present invention is therapeutic. In another embodiment, the protocol is prophylactic. Each possibility represents a separate embodiment of the present invention.

In another embodiment, minor modifications are made to peptides of the present invention without decreasing their affinity for HLA-A*0201 molecules or changing their TCR specificity, utilizing principles well known in the art. “Minor modifications,” in one embodiment, refers to e.g. insertion, deletion, or substitution of one AA, inclusive, or deletion or addition of 1-3 AA outside of the residues between 2 and 9, inclusive. While the computer algorithms described herein are useful for predicting the MHC class I-binding potential of peptides, they have 60-80% predictive accuracy; and thus, the peptides should be evaluated empirically before a final determination of MHC class I-binding affinity is made. Thus, peptides of the present invention are not limited to peptides predicated by the algorithms to exhibit strong MHC class I-binding affinity. The types are modifications that can be made are listed below. Each modification represents a separate embodiment of the present invention.

In another embodiment, a peptide used in the Examples of the present invention is further modified by mutating an anchor residue to an MHC class I preferred anchor residue. In another embodiment, a peptide of the present invention containing an MHC class I preferred anchor residue is further modified by mutating the anchor residue to a different MHC class I preferred residue for that location. The different preferred residue can be any of the preferred residues enumerated herein.

In one embodiment, the anchor residue that is further modified is in the 1 position.

In another embodiment, the anchor residue is in the 2 position. In another embodiment, the anchor residue is in the 3 position. In another embodiment, the anchor residue is in the 4 position. In another embodiment, the anchor residue is in the 5 position. In another embodiment, the anchor residue is in the 6 position. In another embodiment, the anchor residue is in the 7 position. In another embodiment, the anchor residue is in the 8 position. In another embodiment, the anchor residue is in the 9 position. Residues other than 2 and 9 can also serve as secondary anchor residues; therefore, mutating them can improve MHC class I binding. Each possibility represents a separate embodiment of the present invention.

In another embodiment, the peptide is a length variant of a peptide used in the Examples of the present invention. In one embodiment, the length variant is one amino acid (AA) shorter than the peptide disclosed in the Examples. In another embodiment, the shorter peptide is truncated on the N-terminal end. Peptides have been shown to be amenable to truncation on the N-terminal end without changing affinity for HLA-A*0201 molecules, as is well known in the art.

In another embodiment, the length variant is longer than a peptide disclosed in the Examples of the present invention. In another embodiment, the longer peptide is extended on the N-terminal end in accordance with the surrounding bcr-abl sequence. Peptides have been shown to extendable on the N-terminal end without changing affinity for HLA-A*0201 molecules, as is well known in the art. Such peptides are thus equivalents of the peptides disclose in the Examples of the present invention. In another embodiment, the N-terminal extended peptide is extended by one residue.

In another embodiment, the N-terminal extended peptide is extended by two residues. In another embodiment, the N-terminal extended peptide is extended by three residues. In another embodiment, the N-terminal extended peptide is extended by more than three residues. Each possibility represents a separate embodiment of the present invention.

In one embodiment, the longer peptide is extended on the C terminal end in accordance with the surrounding bcr-abl sequence. In another embodiment, the C-terminal extended peptide is extended by one residue. Peptides have been shown to extendable on the C-terminal end without changing affinity for HLA-A*0201 molecules, as is well known in the art. Such peptides are thus equivalents of the peptides disclosed in the Examples of the present invention.

In another embodiment, the C-terminal extended peptide is extended by two residues.

In another embodiment, the C-terminal extended peptide is extended by three residues. In another embodiment, the C-terminal extended peptide is extended by more than three residues. Each possibility represents a separate embodiment of the present invention.

In another embodiment, a truncated peptide of the present invention retains the HLA A0201 anchor residues on the second residue and the C-terminal residue, with a smaller number of intervening residues (e.g. 5) than a peptide disclosed in the Examples of the present invention. In one embodiment, such a truncated peptide is designed by removing one of the intervening residues of one of the above sequences. In another embodiment, the HLA A0201 anchor residues are retained on the second and eighth residues. In another embodiment, the HLA A0201 anchor residues are retained on the first and eighth residues. Each possibility represents a separate embodiment of the present invention.

In another embodiment, an extended peptide of the present invention retains the HLA A0201 anchor residues on the second residue and the C-terminal residue, with a larger number of intervening residues (e.g. 7 or 8) than a peptide disclosed in the Examples of the present invention. In one embodiment, such an extended peptide is designed by adding one or more residues between two of the intervening residues of one of the above sequences. It is well known in the art that residues can be removed from or added between the intervening sequences of HLA A0201 -binding peptides without changing affinity for HLA A0201. Such peptides are thus equivalents of the peptides disclosed in the Examples of the present invention. In another embodiment, the HLA A0201 anchor residues are retained on the second and ninth residues. In another embodiment, the HLA A0201 anchor residues are retained on the first and eighth residues. In another embodiment, the HLA A0201 anchor residues are retained on the two residues separated by six intervening residues. Each possibility represents a separate embodiment of the present invention.

In another embodiment, a peptide of the present invention is homologous to a peptide disclosed in the Examples. The terms “homology,” “homologous,” etc, when in reference to any protein or peptide, refer, in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 2, 4, 5, 7, 9, 10, and 13-16 of greater than 65%. In another embodiment, “homology” refers to identity to one of the above sequences of greater than 70%. In another embodiment, “homology” refers to identity to one of the above sequences of greater than 72%. In another embodiment, “homology” refers to identity to one of the above sequences of greater than 75%. In another embodiment, “homology” refers to identity to one of the above sequences of greater than 78%. In another embodiment, “homology” refers to identity to one of the above sequences of greater than 80%. In another embodiment, “homology” refers to identity to one of the above sequences of greater than 82%. In another embodiment, “homology” refers to identity to one of the above sequences of greater than 83%. In another embodiment, “homology” refers to identity to one of the above sequences of greater than 85%. In another embodiment, “homology” refers to identity to one of the above sequences of greater than 87%. In another embodiment, “homology” refers to identity to a sequence selected from SEQ ID No: 1-41 of greater than 88%. In another embodiment, “homology” refers to identity to one of the above sequences of greater than 90%. In another embodiment, “homology” refers to identity to one of the above sequences of greater than 92%. In another embodiment, “homology” refers to identity to one of the above sequences of greater than 93%. In another embodiment, “homology” refers to identity to one of the above sequences of greater than 95%. In another embodiment, “homology” refers to identity to one of the above sequences of greater than 96%. In another embodiment, “homology” refers to identity to one of the above sequences of greater than 97%. In another embodiment, “homology” refers to identity to one of the above sequences of greater than 98%. In another embodiment, “homology” refers to identity to one of the above sequences of greater than 99%. In another embodiment, “homology” refers to identity to one of the above sequences of 100%. Each possibility represents a separate embodiment of the present invention.

In another embodiment, homology is determined is via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

Each of the above homologues and variants of peptides disclosed in the Examples represents a separate embodiment of the present invention.

In one embodiment, methods of the present invention provide for an improvement in an immune response that has already been mounted by a subject. In one embodiment, methods of the present invention comprise administering the peptide, composition, or vaccine 2 or more times. In another embodiment, the peptides are varied in their composition, concentration, or a combination thereof. In another embodiment, the peptides provide for the initiation of an immune response against an antigen of interest in a subject in which an immune response against the antigen of interest has not already been initiated. In another embodiment, the CTL that are induced proliferate in response to presentation of the peptide on the antigen-presenting cell or cancer cell. It is to be understood that reference to modulation of the immune response may, in another embodiment, involve both the humoral and cell-mediated arms of the immune system, which is accompanied by the presence of Th2 and Th1 T helper cells, respectively, or in another embodiment, each arm individually. For further discussion of immune responses, see, e.g., Abbas et al. Cellular and Molecular Immunology, 3rd Ed., W.B. Saunders Co., Philadelphia, Pa. (1997).

In other embodiments, the methods affecting the growth of a tumor result in (1) the direct inhibition of tumor cell division, or (2) immune cell mediated tumor cell lysis, or both, which leads to a suppression in the net expansion of tumor cells.

Inhibition of tumor growth by either of these two mechanisms can be readily determined by one of ordinary skill in the art based upon a number of well known methods. In one embodiment, tumor inhibition is determined by measuring the actual tumor size over a period of time. In another embodiment, tumor inhibition can be determined by estimating the size of a tumor (over a period of time) utilizing methods well known to those of skill in the art. More specifically, a variety of radiologic imaging methods (e.g., single photon and positron emission computerized tomography; see generally, “Nuclear Medicine in Clinical Oncology,” Winkler, C. (ed.) Springer-Verlag, New York, 1986), can be utilized to estimate tumor size. Such methods can also utilize a variety of imaging agents, including for example, conventional imaging agents (e.g., Gallium-67 citrate), as well as specialized reagents for metabolite imaging, receptor imaging, or immunologic imaging (e.g., radiolabeled monoclonal antibody specific tumor markers). In addition, non-radioactive methods such as ultrasound (see, “Ultrasonic Differential Diagnosis of Tumors”, Kossoff and Fukuda, (eds.), Igaku-Shoin, New York, 1984), can also be utilized to estimate the size of a tumor.

Methods of determining the presence and magnitude of an immune response are well known in the art. In one embodiment, lymphocyte proliferation assays, wherein T cell uptake of a radioactive substance, e.g. ³H-thymidine is measured as a function of cell proliferation. In other embodiments, detection of T cell proliferation is accomplished by measuring increases in interleukin-2 (IL-2) production, Ca2⁺ flux, or dye uptake, such as 3-(4,5-dimethylthiazol-2-yl)-2,5-diphenyl-tetrazolium. Each possibility represents a separate embodiment of the present invention.

In another embodiment, CTL stimulation is determined by means known to those skilled in the art, including, detection of cell proliferation, cytokine production and others. Analysis of the types and quantities of cytokines secreted by T cells upon contacting ligand-pulsed targets can be a measure of functional activity. Cytokines can be measured by ELISA or ELISPOT assays to determine the rate and total amount of cytokine production. (Fujihashi K. et al. (1993) J. Immunol. Meth. 160:181; Tanguay S. and Killion J. J. (1994) Lymphokine Cytokine Res. 13:259).

In one embodiment, CTL activity is determined by ⁵¹Cr-release lysis assay. Lysis of peptide-pulsed ⁵¹Cr-labeled targets by antigen-specific T cells can be compared for target cells pulsed with control peptide. In another embodiment, T cells are stimulated with a peptide of this invention, and lysis of target cells expressing the native peptide in the context of MHC can be determined. The kinetics of lysis as well as overall target lysis at a fixed timepoint (e.g., 4 hours) are used, in another embodiment, to evaluate ligand performance. (Ware C. F. et al. (1983) J. Immunol. 131:1312).

In another embodiment, the peptides utilized in methods and compositions of the present invention comprise a non-classical amino acid such as: 1,2,3,4-tetrahydroisoquinoline-3-carboxylate (Kazmierski et al. (1991) J. Am Chem. Soc. 113:2275-2283); (2S,3S)-methyl-phenylalanine, (2S,3R)-methyl-phenylalanine, (2R,3S)-methylphenylalanine and (2R,3R)-methyl-phenylalanine (Kazmierski and Hruby (1991) Tetrahedron Lett. 32(41): 5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1984) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al.(1993) Int. J. Pep. Protein Res. 42(1):68-77) and ((1992) Acta. Crst., Crystal Struc. Comn. 48(IV): 1239-124).

In another embodiment, a peptide of this invention comprises an AA analog or peptidomimetic, which, in other embodiments, induces or favors specific secondary structures. Such peptides comprises, in other embodiments, the following: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Left. 29:5057-5060); .alpha.-helix inducing analogs (Kemp et al. (1988) Tetrahedron Left. 29:4935-4938); .gamma.-turn inducing analogs (Kemp et al. (1989) J. Org. Chem. 54:109:115); analogs provided by the following references: Nagai and Sato (1985) Tetrahedron Left. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Jones et al. (1988) Tetrahedron Left. 29(31):3853-3856); tretrazol (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garveyet al. (1990) J. Org. Chem. 55(3):936-940. Conformationally restricted mimetics of beta turns and beta bulges, and peptides containing them, are described in U.S. Pat. No. 5,440,013, issued Aug. 8, 1995 to Kahn.

In another embodiment, a peptide of this invention is conjugated to various other molecules, as described hereinbelow, which can be via covalent or non-covalent linkage (complexed), the nature of which varies, in another embodiment, depending on the particular purpose. For example, a peptide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome. U.S. Pat. No. 5,837,249. A peptide of the invention can be complexed covalently or non-covalently with a solid support, a variety of which are known in the art.

In one embodiment, the term “amino acid” refers to a natural or, in another embodiment, an unnatural or synthetic AA, and can include, in other embodiments, glycine, D- or L optical isomers, AA analogs, peptidomimetics, or combinations thereof.

In another embodiment, the present invention provides a composition comprising a peptide of this invention. comprising (a) a bcr-abl-based peptide of the present invention and (b) an adjuvant. In another embodiment, the present invention provides a composition comprising a peptide of this invention. comprising (a) a DNA molecule encoding a bcr-abl-based peptide of the present invention and (b) an adjuvant. In another embodiment, the composition further comprises a pharmaceutically acceptable carrier. In another embodiment, the composition further comprises an adjuvant. In another embodiment, the composition comprises two or more peptides of the present invention. In another embodiment, the composition further comprises any of the additives, compounds, or excipients set forth hereinbelow. Each possibility represents a separate embodiment of the present invention.

In another embodiment, a method or composition of the present invention further comprises an immunogenic carrier associated therewith. In one embodiment, the immunogenic carrier is a protein. In another embodiment, the immunogenic carrier is a peptide. In another embodiment, the immunogenic carrier is an antigen-presenting cell. In another embodiment, the immunogenic carrier is any other type of immunogenic carrier known in the art.

In another embodiment, the immunogenic carrier is keyhole limpet hemocyanin. In another embodiment, the immunogenic carrier is an albumin protein. In another embodiment, the immunogenic carrier is a polyamino acid. In another embodiment, the immunogenic carrier is any other types of immunogenic carrier known in the art. Each possibility represents a separate embodiment of the present invention.

In one embodiment of methods and compositions of the present invention, the adjuvant or immunogenic carrier is linked to the peptide. In another embodiment, the adjuvant or immunogenic carrier is associated with the peptide. In another embodiment, the adjuvant or immunogenic carrier is mixed with the peptide. Each possibility represents a separate embodiment of the present invention.

In another embodiment, the present invention provides a vaccine comprising comprising (a) a bcr-abl-based peptide of the present invention and (b) a pharmaceutically acceptable carrier. In another embodiment, the present invention provides a vaccine comprising comprising (a) a DNA molecule encoding a bcr-abl-based peptide of the present invention and (b) a pharmaceutically acceptable carrier. “Vaccine” refers, in one embodiment, to a material that, when introduced into a subject, elicits a prophylactic or for a particular disease, condition, or symptom of same. In another embodiment, the vaccine elicits a therapeutic response. In another embodiment, the composition further comprises any of the additives, compounds, or excipients set forth hereinbelow, including immunomodulating compounds such as cytokines, adjuvants, etc. Each possibility represents a separate embodiment of the present invention.

In one embodiment, the adjuvant is QS21. In another embodiment, the adjuvant is Freund's incomplete adjuvant. In another embodiment, the adjuvant is aluminum phosphate. In another embodiment, the adjuvant is aluminum hydroxide. In another embodiment, the adjuvant is BCG. In another embodiment, the adjuvant is alum. In another embodiment, the adjuvant is a growth factor (e.g. GM-CSF). In another embodiment, the adjuvant is a cytokine. In another embodiment, the adjuvant is a chemokine. In another embodiment, the adjuvant is an interleukin. In another embodiment, the adjuvant is any other adjuvant known in the art. Each possibility represents a separate embodiment of the present invention.

Methods for synthesizing peptides are well known in the art. In one embodiment, the peptides of this invention are synthesized using an appropriate solid-state synthetic procedure (see for example, Steward and Young, Solid Phase Peptide Synthesis, Freemantle, San Francisco, Calif. (1968); Merrifield (1967) Recent Progress in Hormone Res 23: 451). The activity of these peptides is tested, in other embodiments, using assays as described herein.

In another embodiment, the peptides of this invention are purified by standard methods including chromatography (e.g., ion exchange, affinity, and sizing column chromatography), centrifugation, differential solubility, or by any other standard technique for protein purification. In another embodiment, immuno-affinity chromatography is used, whereby an epitope is isolated by binding it to an affinity column comprising antibodies that were raised against that peptide, or a related peptide of the invention, and were affixed to a stationary support.

In another embodiment, affinity tags such as hexa-His (Invitrogen), Maltose binding domain (New England Biolabs), influenza coat sequence (Kolodziej et al. (1991) Meth. Enzymol. 194:508-509), glutathione-S-transferase, or others, are attached to the peptides of this invention to allow easy purification by passage over an appropriate affinity column. Isolated peptides can also be physically characterized, in other embodiments, using such techniques as proteolysis, nuclear magnetic resonance, and x-ray crystallography.

In another embodiment, the peptides of this invention are produced by in vitro translation, through known techniques, as will be evident to one skilled in the art. In another embodiment, the peptides are differentially modified during or after translation, e.g., by phosphorylation, glycosylation, cross-linking, acylation, proteolytic cleavage, linkage to an antibody molecule, membrane molecule or other ligand, (Ferguson et al. (1988) Ann. Rev. Biochem. 57:285-320).

In one embodiment, the peptides of this invention further comprise a detectable label, which in one embodiment, is fluorescent, or in another embodiment, luminescent, or in another embodiment, radioactive, or in another embodiment, electron dense. In other embodiments, the dectectable label comprises, for example, green fluorescent protein (GFP), DS-Red (red fluorescent protein), secreted alkaline phosphatase (SEAP), beta-galactosidase, luciferase, 32P, 125I, 3H and 14C, fluorescein and its derivatives, rhodamine and its derivatives, dansyl and umbelliferone, luciferin or any number of other such labels known to one skilled in the art. The particular label used will depend upon the type of immunoassay used.

In another embodiment, a peptide of this invention is linked to a substrate, which, in one embodiment, serves as a carrier. In one embodiment, linkage of the peptide to a substrate serves to increase an elicited an immune response.

In one embodiment, peptides of this invention are linked to other molecules, as described herein, using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and -ethyl-3-(4-azonia-44-dimethylpentyl) carbodiimide.

In other embodiments, the cross-linking agents comprise cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound can be used. Also envisioned, in other embodiments, are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

In other embodiments, the homo-bifunctional cross-linking agents include the bifunctional N-hydroxysuccinimide esters dithiobis(succinimidylpropionate), disuccinimidyl suberate, and disuccinimidyl tartarate; the bifunctional imido-esters di methyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

In other embodiments, hetero-bifunctional cross-linking agents used to link the peptides to other molecules, as described herein, include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(.gamma.-maleimidobutyryloxy)succinimide ester), MPBH (4-(4-N-maleimidopohenyl) butyric acid hydrazide), M2C2H (4-(N-maleimidomethyl) cyclohexane-1-carboxyl-hydrazide), SMPT (succinimidyloxycarbonyl-a-methyl-a-(2-pyridyldithio)toluene), and SPDP (N-succinimidyl 3-(2-pyridyldithio)propionate).

In another embodiment, the peptides of the invention are formulated as non-covalent attachment of monomers through ionic, adsorptive, or biospecific interactions. Complexes of peptides with highly positively or negatively charged molecules can be accomplished, in another embodiment, through salt bridge formation under low ionic strength environments, such as in deionized water. Large complexes can be created, in another embodiment, using charged polymers such as poly-(L-glutamic acid) or poly-(L-lysine), which contain numerous negative and positive charges, respectively. In another embodiment, peptides are adsorbed to surfaces such as microparticle latex beads or to other hydrophobic polymers, forming non-covalently associated peptide-superantigen complexes effectively mimicking cross-linked or chemically polymerized protein, in other embodiments. In another embodiment, peptides are non-covalently linked through the use of biospecific interactions between other molecules. For instance, utilization of the strong affinity of biotin for proteins such as avidin or streptavidin or their derivatives could be used to form peptide complexes. The peptides, according to this aspect, and in one embodiment, can be modified to possess biotin groups using common biotinylation reagents such as the N-hydroxysuccinimidyl ester of D-biotin (NHS-biotin), which reacts with available amine groups.

In another embodiment, the peptides are linked to carriers. In another embodiments, the peptides are any that are well known in the art, including, for example, thyroglobulin, albumins such as human serum albumin, tetanus toxoid, polyamino acids such as poly (lysine:glutamic acid), influenza, hepatitis B virus core protein, hepatitis B virus recombinant vaccine and the like. Each possibility represents a separate embodiment of the present invention.

In another embodiment, the peptides of this invention are conjugated to a lipid, such as P3 CSS. In another embodiment, the peptides of this invention are conjugated to a bead.

In another embodiment, the compositions of this invention further comprise immunomodulating compounds. In other embodiments, the immunomodulating compound is a cytokine, chemokine, or complement component that enhances expression of immune system accessory or adhesion molecules, their receptors, or combinations thereof. In some embodiments, the immunomodulating compound include interleukins, for example interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components, or combinations thereof. In other embodiments, the immunomodulating compound stimulate expression, or enhanced expression of OX40, OX40L (gp34), lymphotactin, CD40, CD40L, B7.1, B7.2, TRAP, ICAM-1, 2 or 3, cytokine receptors, or combination thereof.

In another embodiment, the immunomodulatory compound induces or enhances expression of co-stimulatory molecules that participate in the immune response, which include, in some embodiments, CD40 or its ligand, CD28, CTLA-4 or a B7 molecule. In another embodiment, the immunomodulatory compound induces or enhances expression of a heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), or an intracellular adhesion molecule 1 (ICAM-1) (Van R. H. (1992) Cell 71:1065-1068), which may assist co-stimulation by interacting with their cognate ligands on the T cells.

In another embodiment, the composition comprises a solvent, including water, dispersion media, cell culture media, isotonic agents and the like. In one embodiment, the solvent is an aqueous isotonic buffered solution with a pH of around 7.0. In another embodiment, the composition comprises a diluent such as water, phosphate buffered saline, or saline. In another embodiment, the composition comprises a solvent, which is non-aqueous, such as propyl ethylene glycol, polyethylene glycol and vegetable oils.

In another embodiment, the composition is formulated for administration by any of the many techniques known to those of skill in the art. For example, this invention provides for administration of the pharmaceutical composition parenterally, intravenously, subcutaneously, intradermally, intramucosally, topically, orally, or by inhalation.

In another embodiment, the vaccine comprising a peptide of this invention further comprises a cell population, which, in another embodiment, comprises lymphocytes, monocytes, macrophages, dendritic cells, endothelial cells, stem cells or combinations thereof, which, in another embodiment are autologous, syngeneic or allogeneic, with respect to each other. In another embodiment, the cell population comprises a peptide of the present invention. In another embodiment, the cell population takes up the peptide. Each possibility represents a separate embodiment of the present invention.

In one embodiment, the cell populations of this invention are obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells can be obtained. In one embodiment, the cell populations are obtained from human sources, which are, in other embodiments, from human fetal, neonatal, child, or adult sources. In another embodiment, the cell populations of this invention are obtained from animal sources, such as, for example, porcine or simian, or any other animal of interest. In another embodiment, the cell populations of this invention are obtained from subjects that are normal, or in another embodiment, diseased, or in another embodiment, susceptible to a disease of interest.

In another embodiment, the cell populations of this invention are separated via affinity-based separation methods. Techniques for affinity separation include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques include the use of fluorescence activated cell sorters, which can have varying degrees of sophistication, such as multiple color channels, low angle and obtuse light scattering detecting channels, impedance channels, etc. It is to be understood that any technique that enables separation of the cell populations of this invention may be employed, and is to be considered as part of this invention.

In one embodiment, the dendritic cells are from the diverse population of morphologically similar cell types found in a variety of lymphoid and non-lymphoid tissues, qualified as such (Steinman (1991) Ann. Rev. Immunol. 9:271-296). In one embodiment, the dendritic cells used in this invention are isolated from bone marrow, or in another embodiment, derived from bone marrow progenitor cells, or, in another embodiment, from isolated from/derived from peripheral blood, or in another embodiment, derived from, or are a cell line.

In one embodiment, the cell populations described herein are isolated from the white blood cell fraction of a mammal, such as a murine, simian or a human (See, e.g., WO 96/23060). The white blood cell fraction can be, in another embodiment, isolated from the peripheral blood of the mammal.

Methods of isolating dendritic cells are well known in the art. In one embodiment, the DC are isolated via a method which includes the following steps: (a) providing a white blood cell fraction obtained from a mammalian source by methods known in the art such as leukophoresis; (b) separating the white blood cell fraction of step (a) into four or more subfractions by countercurrent centrifugal elutriation; (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c); and (e) collecting the enriched fraction of step (d), preferably at about 4° C.

In another embodiment, the dendritic cell-enriched fraction is identified by fluorescence-activated cell sorting, which identifies at least one of the following markers: HLA-DR, HLA-DQ, or B7.2, and the simultaneous absence of the following markers: CD3, CD 14, CD 16, 56, 57, and CD 19, 20.

In another embodiment, the cell population comprises lymphocytes, which are, in one embodiment, T cells, or in another embodiment, B cells. The T cells are, in other embodiments, characterized as NK cells, helper T cells, cytotoxic T lymphocytes (CTL), TILs, naïve T cells, or combinations thereof. It is to be understood that T cells which are primary, or cell lines, clones, etc. are to be considered as part of this invention. In one embodiment, the T cells are CTL, or CTL lines, CTL clones, or CTLs isolated from tumor, inflammatory, or other infiltrates.

In another embodiment, hematopoietic stem or early progenitor cells comprise the cell populations used in this invention. In one embodiment, such populations are isolate or derived, by leukaphoresis. In another embodiment, the leukaphoresis follows cytokine administration, from bone marrow, peripheral blood (PB) or neonatal umbilical cord blood. In one embodiment the stem or progenitor cells are characterized by their surface expression of the surface antigen marker known as CD34+, and exclusion of expression of the surface lineage antigen markers, Lin-.

In another embodiment, the subject is administered a peptide, composition or vaccine of this invention, in conjunction with bone marrow cells. In another embodiment, the administration together with bone marrow cells embodiment follows previous irradiation of the subject, as part of the course of therapy, in order to suppress, inhibit or treat cancer in the subject.

In one embodiment, the phrase “contacting a cell” or “contacting a population” refers to a method of exposure, which may be direct or indirect. In one method such contact comprises direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described herein.

In one embodiment, CTL generation of methods of the present invention is accomplished in vivo, and is effected by introducing into a subject an antigen presenting cell contacted in vitro with a peptide of this invention (See for example Paglia et al. (1996) J. Exp. Med. 183:317-322).

In another embodiment, the peptides of methods and compositions of the present invention are delivered to antigen-presenting cells (APC).

In another embodiment, the peptides are delivered to APC in the form of cDNA encoding the peptides. In one embodiment, the term “antigen-presenting cells” refers to dendritic cells (DC), monocytes/macrophages, B lymphocytes or other cell type(s) expressing the necessary MHC/co-stimulatory molecules, which effectively allow for T cell recognition of the presented peptide. In another embodiment, the APC is a cancer cell. Each possibility represents a separate embodiment of the present invention.

In another embodiment, the CTL are contacted with two or more antigen-presenting cell populations. In another embodiment, the two or more antigen presenting cell populations present different peptides. Each possibility represents a separate embodiment of the present invention.

In another embodiment, techniques that lead to the expression of antigen in the cytosol of APC (e.g. DC) are used to deliver the peptides to the APC. Methods for expressing antigens on APC are well known in the art. In one embodiment, the techniques include (1) the introduction into the APC of naked DNA encoding a peptide of this inveniton, (2) infection of APC with recombinant vectors expressing a peptide of this invention, and (3) introduction of a peptide of this invention into the cytosol of an APC using liposomes. (See Boczkowski D. et al. (1996) J. Exp. Med. 184:465-472; Rouse et al. (1994) J. Virol. 68:5685-5689; and Nair et al. (1992) J. Exp. Med. 175:609-612).

In another embodiment, foster antigen presenting cells such as those derived from the human cell line 174xCEM.T2, referred to as T2, which contains a mutation in its antigen processing pathway that restricts the association of endogenous peptides with cell surface MHC class I molecules (Zweerink et al. (1993) J. Immunol. 150:1763-1771), are used, as exemplified herein.

In one embodiment, as described herein, the subject is exposed to a peptide, or a composition/cell population comprising a peptide of this invention, which differs from the native protein expressed, wherein subsequently a host immune cross-reactive with the native protein/antigen develops.

In one embodiment, the subject, as referred to in any of the methods or embodiments of this invention is a human. In other embodiments, the subject is a mammal, which may be a mouse, rat, rabbit, hamster, guinea pig, horse, cow, sheep, goat, pig, cat, dog, monkey, or ape. Each possibility represents a separate embodiment of the present invention.

The peptides of this invention may, in one embodiment, stimulate an immune response that results in tumor cell lysis. In one embodiment, the method of treating a subject with cancer entails directly administering a peptide of this invention, or in another embodiment, the method entails administering the peptide in a composition, or a vaccine comprising other cells, which, in another embodiment, may be immune cells which are autologous, syngeneic or allogeneic to the subject. In another embodiment, the peptide is first contacted with an antigen presenting cell in vitro, whereby administration of the antigen presenting cell stimulates an immune response to the cancer in the subject.

In one embodiment, any of the methods described herein is used to elicit CTL, which are elicited in vitro. In another embodiment, the CTL are elicited ex-vivo. In another embodiment, the CTL are elicited in vitro. The resulting CTL, may, in another embodiment, be administered to the subject, and thereby treat the condition associated with the peptide, or an expression product comprising the peptide or a homologue thereof. Each possibility represents a separate embodiment of the present invention.

In another embodiment, the method entails introduction of the genetic sequence that encodes the peptides of this invention. In one embodiment, the method comprises administering to the subject a vector comprising a nucleotide sequence, which encodes a peptide of the present invention (Tindle, R. W. et al. Virology (1994) 200:54). In another embodiment, the method comprises administering to the subject naked DNA which encodes a peptide, or in another embodiment, two or more peptides of this invention (Nabel, et al. PNAS-USA (1990) 90: 11307). In another embodiment, multi-epitope, analogue-based cancer vaccines are utilized (Fikes et al, ibid). Each possibility represents a separate embodiment of the present invention.

Nucleic acids can be administered to a subject via any means as is known in the art, including parenteral or intravenous adminstration, or in another embodiment, by means of a gene gun. In another embodiment, the nucleic acids are administered in a composition, which may, in other embodiments, correspond to any embodiment listed herein.

The DNA molecule encoding a bcr-abl-based peptide of the present is, in one embodiment, DNA is contained in a vector. In another embodiment, the DNA molecule is contained in an antigen presenting cell. In another embodiment, the DNA molecule is associated with any other means known in the art of facilitating expressing of a DNA molecule by a mammalian cell. Each possibility represents a separate embodiment of the present invention.

Vectors for use according to methods of this invention can comprise any vector that facilitates or allows for the expression of a peptide of this invention. Vectors comprise, in some embodiments, attenuated viruses, such as vaccinia or fowlpox, such as described in, e.g., U.S. Pat. No.4,722,848, incorporated herein by reference. In another embodiment, the vector is BCG (Bacille Calmette Guerin), such as described in Stover et al. (Nature 351:456-460 (1991)). A wide variety of other vectors useful for therapeutic administration or immunization of the peptides of the invention, e.g., Salmonella typhi vectors and the like, will be apparent to those skilled in the art from the description herein.

In one embodiment, the vector further encodes for an immunomodulatory protein or peptide, as described herein. In another embodiment, the subject is administered an additional vector encoding same, concurrent, prior to or following administration of the vector encoding a peptide of this invention to the subject.

In addition to the in vivo methods for determining tumor inhibition discussed above, a variety of in vitro methods can be utilized in order to predict in vivo tumor inhibition. Representative examples include lymphocyte mediated anti-tumor cytolytic activity determined for example, by a 51 Cr release assay (Examples), tumor dependent lymphocyte proliferation (Ioannides, et al., J. Immunol. 146(5): 1700-1707, 1991), in vitro generation of tumor specific antibodies (Herlyn, et al., J. Immunol. Meth. 73:157-167, 1984), cell (e.g., CTL, helper T-cell) or humoral (e.g., antibody) mediated inhibition of cell growth in vitro (Gazit, et al., Cancer Immunol Immunother 35:135-144, 1992), and, for any of these assays, determination of cell precursor frequency (Vose, Int. J. Cancer 30:135-142 (1982), and others.

In another embodiment, the subject is administered a peptide following previous administration of chemotherapy to the subject. In another embodiment, the subject has been treated with imatinib. In another embodiment, the cancer in the subject is resistant to imatinib treatment.

In another embodiment, according to these aspects of the invention, methods of suppressing tumor growth indicate a growth state that is curtailed compared to growth without contact with, or exposure to a peptide of this invention. Tumor cell growth can be assessed by any means known in the art, including, but not limited to, measuring tumor size, determining whether tumor cells are proliferating using a 3H-thymidine incorporation assay, or counting tumor cells. “Suppressing” tumor cell growth refers, in other embodiments, to slowing, delaying, or stopping tumor growth, or to tumor shrinkage. Each possibility represents a separate embodiment of the present invention.

In another embodiment, the peptides, compositions and vaccines of this invention are administered to a subject, or utilized in the methods of this invention, in combination with other anti-cancer compounds and chemotherapeutics, including monoclonal antibodies directed against alternate cancer antigens, or, in another embodiment, epitopes that consist of an AA sequence which corresponds to, or in part to, that from which the peptides of this invention are derived.

### EXPERIMENTAL DETAILS SECTION

### Materials and Experimental Methods

Subjects

Informed consent was obtained for all subjects in the study. Blood samples were obtained from accelerated phase and chronic phase patients.

Peptides

Peptides were synthesized by Genemed Synthesis Inc, CA using fluorenylmethoxycarbonyl chemistry, solid phase synthesis and purified by high pressure liquid chromatography (HPLC). The quality of the peptides was assessed by HPLC analysis, and the expected molecular weight was observed using matrix-assisted laser desorption mass spectrometry. Peptides were sterile and >90% pure. The peptides were dissolved in DMSO and diluted in phosphate-buffered saline (PBS; pH 7.4) or saline at a concentration of 5 mg/ml and were stored at −80° C.

Cell Lines

Cell lines were cultured in RPMI 1640 medium supplemented with 5% FCS, penicillin, streptomycin, 2 mM glutamine and 2-mercaptoethanol at 37° C. in humidified air containing 5% CO₂. SKLY-16 is a human B cell lymphoma expressing HLA A0201, and T2 is a human cell line lacking TAP1 and TAP2 and therefore unable to present peptides derived from cytosolic proteins.

T2 Assay for Peptide Binding and Stabilization of HLA A0201 Molecules

T2 cells (TAP-, HLA-A0201⁺) were incubated overnight at 27° C. at a concentration of 1×10⁶ cells/ml in FCS-free RPMI medium supplemented with 5 μg/ml human β₂microglobulin (Sigma, St Louis, Mo.) in the absence (negative control) or presence of either a positive reference tyrosinase peptide or test peptides at various final concentrations (50, 10, 1, and 0.1 μg/ml). Following a 4-hour incubation with 5 μg/ml brefeldin A (Sigma), T2 cells were labeled for 30 minutes at 4° C. with a saturating concentration of anti-HLA-A2.1 (BB7.2) monoclonal antibody (mAb), then washed twice. The cells were then incubated for 30 minutes at 4° C. with a saturating concentration of FTrC-conjugated goat IgG F(ab′)2 anti-mouse Ig (Caltag, South San Francisco, Calif.), washed twice, fixed in PBS/1% paraformaldehyde and analyzed using a FACS Calibur® cytofluorometer (Becton Dickinson, Immunocytometry systems, San Jose, Calif.).

The mean intensity of fluorescence (MIF) observed for each peptide concentration (after dividing of the MIF observed without peptide) was used as an estimate of peptide binding and expressed as a fluorescence index. Stabilization assays were performed similarly. Following initial evaluation of peptide binding at time 0, cells were washed in RPMI complete medium to remove free peptides and incubated in the continuous presence of 0.5 μg/ml brefeldin-A for 2, 4, 6 or 8 hours.

The amount of stable peptide-HLA-A2.1 complexes was estimated as described above by indirect immunofluorescence analysis. The half life of complexes is the time required for a 50% reduction of the time 0 MIF value.

Human Dendritic Cell Isolation

Peripheral blood mononuclear cells (PBMC) from HLA-A0201 positive healthy donors and chronic myeloid leukemia (CML) patients were isolated by Ficoll-density centrifugation. Peripheral blood dendritic cells (DCs) were generated as follows: Monocyte-enriched PBMC fractions were isolated, using a plastic adherence technique, from total PBMC. The plastic-adherent cells were cultured further in RPMI 1640 medium supplemented with 1-5% autologous plasma, 1000 U/mL recombinant human interleukin (IL)-4 (Schering-Plough, NJ), and 1000 U/mL recombinant human granulocyte-macrophage colony-stimulating factor (GM-CSF) (Immunex, Seattle). On days 2 and 4 of incubation, part of the medium was exchanged for fresh culture medium supplemented with IL-4 and GM-CSF, and culture was continued. On day 6, half of the medium was exchanged for culture medium supplemented with IL-4, GM-CSF, and 10 ng/mL recombinant human tumor necrosis factor (TNF)-alpha (R&D system) and 500 ng/ml of trimeric soluble CD40L (Immunex, Seattle). On day 9, the cells were harvested and used as monocyte-derived DC for antigen stimulation. These cells expressed DC-associated antigens, such as CD80, CD83, CD86, and HLA class I and class II on their cell surfaces (data not shown).

In Vitro Immunization and Human T Cell Cultures

T lymphocytes were isolated from the same donors by use of negative selection by depletion with an anti-CD11b, anti-CD56 and CD19 MAb (Miltenyi, CA). A total of 1×10⁶ pure T lymphocytes were cultured with 1×10⁵ autologous DC in RPMI 1640 medium supplemented with 5% heat-inactivated human autologous plasma with bcr-abl synthetic peptides at a concentration of 10 μg/mL and β₂ microglobulin at 2 μg/ml in 24-well plates in the presence of5-10 ng/mL recombinant human IL-7 (Genzyme) and 0.1 ng/ml IL-12. After culture for 3 days 20 U/ml of IL-2 was added. After 10 days, 1×10⁶ cells were stimulated again by adding 2×10⁵ autologous, magnetically isolated CD 14⁺ monocytes together with 10 ng/ml of IL-7 and 20 U/ml IL-2 and peptide at a concentration of 10 μg/mL. In some cases, as indicated, after culture for another 7 days, the cells were stimulated a third time, in the same manner. After the second or third stimulation, CD8 T cells were magnetically isolated and cytotoxicity and gamma-IFN (interferon) secretion of these cells was then examined.

Gamma Interferon ELISPOT

HA-Multiscreen® plates (Millipore, Burlington, Mass.) were coated with 100 μl of mouse-anti-human IFN-gamma antibody (10 μg/ml; clone 1-DLK, Mabtech, Sweden) in PBS, incubated overnight at 4° C., washed with PBS to remove unbound antibody and blocked with RPMI/autologous plasma for 1 hour at 37° C. Purified CD8⁺ T cells (more than 95% pure) were plated at a concentration of 1×10⁵/well. T cells were stimulated with 1×10⁴T2 cells per well pulsed with 10 μg/ml of β₂-microglobulin and either 50 μg/ml of test peptide, positive control influenza matrix peptide GILGFVFTL (SEQ ID No: 63; Bocchia M, Korontsvit T et al, Blood 1996; 87(9): 3587-92), or irrelevant control peptide, HILA A24 consensus motif (SEQ ID No: 213) at a final volume of 100-200 μl/well. Control wells contained T2 cells with or without CD8⁺ cells. Additional controls included medium or CD8⁺ alone plus PBS/5% DMSO diluted according to the concentrations of peptides used for pulsing T2 cells. After incubation for 20 h (hours) at 37° C., plates were extensively washed with PBS/0.05% Tween and 100 μl/well biotinylated detection antibody against human IFN-γ (2 μg/ml; clone 7-B6-1, Mabtech, Sweden) were added. Plates were incubated for an additional 2 h at 37° C. and spot development was performed. Spot numbers were automatically determined with the use of a computer-assisted video image analyzer with KS ELISPOT 4.0 software (Carl Zeiss Vision, Germany).

Cytotoxicity Assay

The presence of specific CTLs was measured in a standard 4 h-chromium release assay as follows. 4×10⁶ targets were labeled with 300 μCi of Na₂⁵¹CrO₊ (NEN Life Science Products, Inc. Boston, Mass.) for 1 hour at 37° C. After washing, cells at 2×10⁶/ml concentration were incubated with or without synthetic peptides at a concentration of 10 μg/ml for 2 h at 20° C. in presence of β₂ microglobulin at 3 μg/ml. After washing by centrifugation, target cells were resuspended in complete media at 5×10⁴ cells per ml and plated in a 96 well U-bottom plate (Becton Dickinson®, NY) at 5×10³cells per well with effector cells at effector to target (E/T) ratios ranging from 100:1 to 10:1. Plates were incubated for 5 hours at 37° C. in 5% CO₂. Supernatant fluids were harvested and radioactivity was measured in a gamma counter. Percent specific lysis was determined from the following formula: 100×[(experimental release−spontaneous release)/(maximum release−spontaneous release)]. Maximum release was determined by lysis of targets in 2.5% Triton X-100.

### EXAMPLE 1

### Identification and Generation of bcr-abl Breakpoint Peptides with a High Probability of HLA A0201 Binding

Peptides with potential CTL epitopes can be predicted by means of a peptide library-based scoring system for MHC class I-binding peptides. AA sequences of the human b3a2 and b2a2 fusion proteins were scanned for peptides with potential binding capacity for HLA A0201, a subtype encompassing 95% of the HLA-A02 allele. HLA-A0201 is expressed in about 40% of the Caucasian population.

Single or double AA substitutions were introduced at HLA A0201 preferred residues (positions 1, 2, 6 and 9, see underlined residues in Table 1) of a peptide that does not exhibit the consensus HLA 0201 binding motifs but has weak avidity to MHC, to yield sequences that had comparatively high binding scores predicted for HLA A0201 molecules. Substitutions were determined using the software of the Bioinformatics & Molecular Analysis Section (National Institutes of Health, Washington, D.C.) (Parker K C, et al. J Immunol 1994; 152(1):163-75; available at http:/fbimas.dcrt.nih.gov/cgi-bin/molbio/ken_parker_comboform), which ranks 9-mer or 10-mer peptides on a predicted half life dissociation coefficient from HLA class I molecules. Several analogue peptides were designed whereby one or both anchor amino acids or additional amino acids adjacent to anchor amino acids were modified. The predicted half life for binding to HLA A0201 was greater than 240 minutes in four synthetic peptides and less than 240 in seven. All the native peptides were predicted to have a half life of less than one minute. Most substitutions affected the primary or secondary anchor motifs (leucine in position 2 or valine in position 9 or 6) but in some cases, a tyrosine was substituted in position 1. This substitution has been shown to stabilize the binding of the position 2 anchor residue. Also depicted in Table 1 are the predicted half lives according to another software program, SYFPEITHI (Rammensee HG, et al., Immunogenetics 1995; 41(4): 178-228; available at http://syfpeithi.bmiheidelberg.com).

TABLE 1

The AA sequences of native breakpoint pep-
tides and synthetic analogues and their
predicted score for binding to HLA A0201,
generated by two BIMAS and SYFPEITHI.

| | | | | |
|---|---|---|---|---|
| | | | | SEQ |
| | | BIMAS | SYFPEITHI | ID |
| Name/type | Sequence | score | score | NO: |

p210-b3a2

| | | | | |
|---|---|---|---|---|
| CMLA2 native | SSKALQRPV | 0.003 | 12 | 1 |
| p210F (analogue) | YLKALQRPV | 2.240 | 22 | 2 |
| CMLA3 native | KQSSKALQR | 0.005 | 3 | 3 |
| p210A (analogue) | KQSSKALQV | 24.681 | 13 | 4 |
| p210B (analogue) | KLSSKALQV | 243.432 | 23 | 5 |
| p210Cn native | KALQRPVAS | 0.013 | 10 | 6 |
| p210C (analogue) | KLLQRPVAV | 900.689 | 26 | 7 |
| p210Dn native | TGFKQSSKA | 0.120 | 7 | 8 |
| p210D (analogue) | TLFKQSSKV | 257.342 | 23 | 9 |
| p210E (analogue) | YLFKQSSKV | 1183.775 | 25 | 10 |

p210-b2a2

| | | | | |
|---|---|---|---|---|
| b2a2A native | LTINKEEAL | 0.247 | 20 | 11 |
| b2a2 A1 (analogue) | LLINKEEAL | 17.795 | 26 | 12 |
| b2a2 A2 (analogue) | LTINKVEAL | 21.996 | 24 | 13 |
| b2a2 A3 (analogue) | YLINKEEAL | 48.151 | 26 | 14 |
| b2a2 A4 (analogue) | YLINKEEAV | 156.770 | 26 | 15 |
| b2a2 A5 (analogue) | YLINKVEAL | 110.747 | 30 | 16 |
| HLA A24 consensus | VYFFLPDHL | | | 17 |
| peptide | | | | |
| positive control | GILGFVFTL | | | 18 |
| influenza matrix | | | | |
| peptide | | | | |

Residues in bold (K in the b3a2 and E in b2a2) represent the amino acid at the fusion breakpoint. Residues underlined represent modifications from the native sequence.

### EXAMPLE 2

### Binding of HLA-A0201 by Selected Peptides

For peptides to be immunogenic, in an MHC class I-restricted context, they require the capacity to bind and stabilize MHC class I molecules on the live cell surface. Since the computer prediction models above have 60-80% predictive accuracy, direct measurement of the strength of the interaction between the peptides and the HLA-A0201 molecule was performed, with a conventional binding and stabilization assay that uses the antigen-transporting deficient (TAP2 negative) HLA-A0201 human T2 cells.

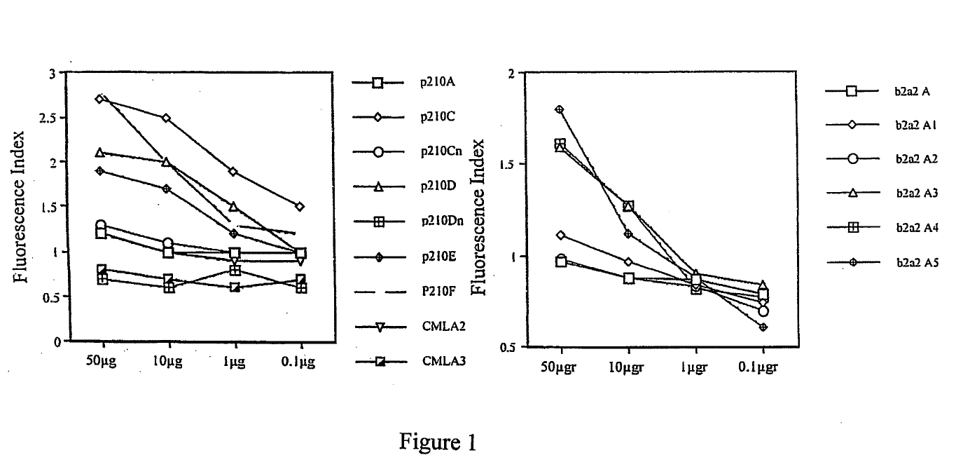

T2 cells lack TAP function and consequently are defective in properly loading class I molecules with antigenic peptides generated in the cytosol. The association of exogenously added peptides with thermolabile, empty HLA-A2 molecules stabilizes them and results in an increase in the level of surface HLA-A0201 recognizable by specific mAb such as BB7.2. Seven out eleven peptides designed to have higher binding scores exhibited a relatively high binding affinity for HLA A0201 molecules as measured by the T2 assay (FIG. 1, left panel). A rough correlation between binding scores and binding affinity was established, thus indicating the utility of the computer generated binding scores for predicting peptides that will bind to MHC class I molecules on live cells.

Some of these peptides demonstrated the same order of binding affinity as that of the influenza matrix viral antigen, which are among the most potent known antigens for CTL induction. In only four cases was a good correlation between computer predicted half-life and T2 stabilization not found.

One of the peptides derived from b3a2, p210C, was mutated from a native peptide that did not have a good prediction score. Nevertheless, the native sequence was able to bind HLA A0201 weakly and at the same level that the previously described CMLA2 peptide. To design p210C, a neutral alanine was substituted for a leucine in position two and a serine was substituted for a valine in position nine. p210C has a high BIMAS score that correlated with T2 binding assay data (FIG. 1, left panel). p210F is a peptide derived from a sequence previously described (Yotnda P, et al., J Clin Invest 1998; 101(10):2290-6), CMLA2, shown to be a weak binder in the T2 assay. In this case the two serines in position one and two were substituted for a tyrosine and a leucine, respectively, with the intent of increasing peptide binding and stabilization to HLA A0201, while retaining the amino-acids for the TCR interaction. The BIMAS prediction was increased 700-fold, and high avidity for HLA A0201 molecules was demonstrated by binding to T2 cells. Of the peptides derived from b2a2, all were generated from a peptide that was not predicted to binding avidly to HLA A0201. Three new synthetic peptides, b2a2 A3-A5 (Table 1) bound well to HLA A0201 molecules (FIG. 1, right panel). These three peptides have a tyrosine-leucine sequence substitution at position 1 and 2 and also a valine substitution in position 6 or 9 that are reflected in increased binding to HLA A0201.

These results show that heteroclitic peptides of the present invention exhibit increased MHC molecule binding, and therefore are likely to have increased immunogenicity.

### EXAMPLE 3

### Peptide Analogue Dissociation from HLA A0201

The immunogenicity of peptide antigens is also related to their low dissociation rate from MHC molecule-peptide complexes. The stability of complexes formed between HLA-A0201 and the b3a2 analogue peptides was therefore assayed with T2 cells, as a function of time. Overnight incubation of T2 cells with saturating amounts of HLA-A0201 binding peptides and humanβ₂ microglobulin resulted in increased surface HLA-A0201 expression. After removal of unbound peptide and addition of brefeldin A to inhibit protein synthesis, the number of HLA-A0201 molecules remaining at the T2 cell surface was determined. The stability of each peptide/HLA-A0201 complex was then normalized relative to that observed for the tyrosinase D peptide or HIV gag peptide (peptides with known high affinity and half life). HLA-A0201 complexes with p210C, p210D, p210E and p210F formed complexes that were stable over 6-8 hours. In contrast, p210A and p210B were less stable, reaching background levels in less than 1 hour of incubation.

These results confirm the results of the previous Example, showing that heteroclitic peptides of the present invention exhibit increased MHC molecule binding.

### EXAMPLE4

### P210 Peptide Stimulation of CD8 Immune Responses; T Cells Generated by Synthetic Analogues Recognized Native Sequences

Peptide affinity for MHC molecules is necessary for immunogenicity; however its ability to induce reactive precursor T cells with cognate T cell receptors is necessary, as well. Using an optimized T cell-expansion system, with monocyte derived DC, CD 14⁺ cells as APC, and purified CD3⁺ T cells, synthetic b3a2 and b2a2 analogues were evaluated for their ability to stimulate peptide-specific CTLs. Cells from ten healthy HLA A0201 donors and 4 patients with chronic phase CML were assayed. The peptides used were heteroclitic peptides p210A, p210B, p210C, p210D, and p210E, and CMLA3, p210Cn, p201Dn, and CMLA2, the native sequences corresponding to p210A-B, p210C, p210D, and p210E, respectively (Table 1).

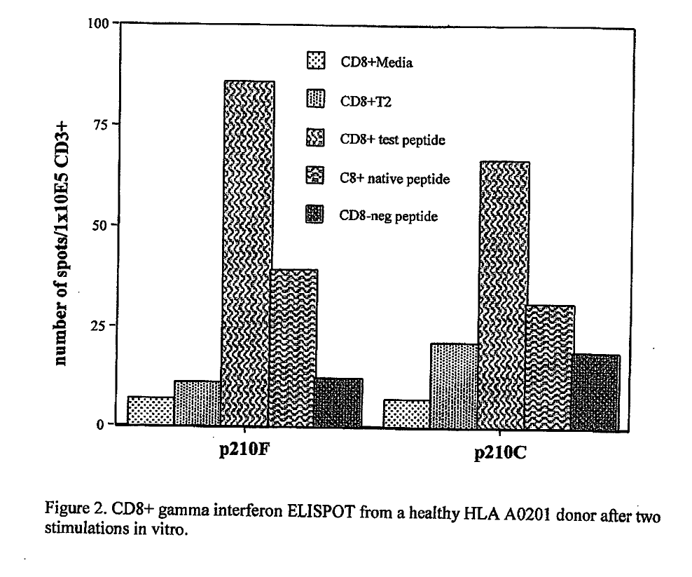

Cells from 5/10 healthy donors responded to immunization, generating T cells that secreted IFN-gamma when challenged with peptide-pulsed T2 cells as targets. p210C and p210F generated the most consistent and significant immune-responses (FIG. 2); p210D and p210E also produced an immune response in some donors tested. Responses were observed after the second or third round of peptide stimulation, either after CD8⁺ isolation or in CD3⁺ T cells not subject to further purification. Spot numbers were consistently higher with peptides that bound with higher affinity to HLA 0201 molecules in the T2 assay. By contrast, no immune response was generated against p210A and p210B, consistent with their reduced affinity for MHC.

In addition, the T cell elicited by p210C and p210F vaccination were able to recognize their respective native sequences (FIG. 2). For example, the peptide CMLA2, the native sequence corresponding to p210F, is a weak MHC binder, and is expressed in the surface of CML blasts.

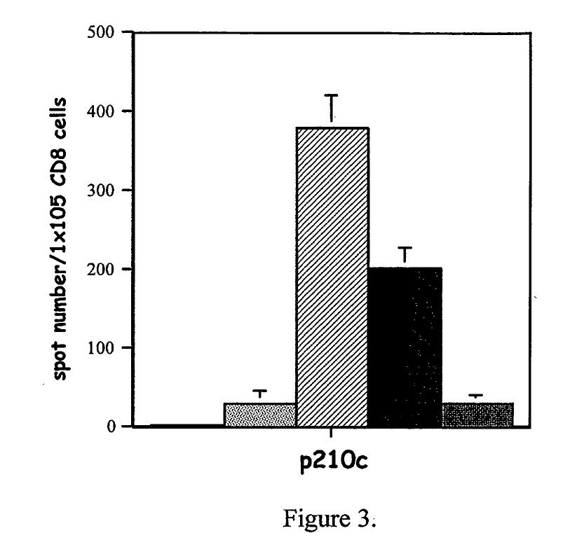

Immune responses to the heteroclitic peptide p210C were also observed in two of the CML patients. After two rounds of stimulation with p210C, CD8⁺ cells recognized T2 pulsed with the synthetic peptide with a frequency of nearly 400 spot-forming cells (SCF) per 1×10⁵ cells, and recognized the native peptide on T2 cells with a frequency of 200 SFC per 1×10⁸ (FIG. 3).

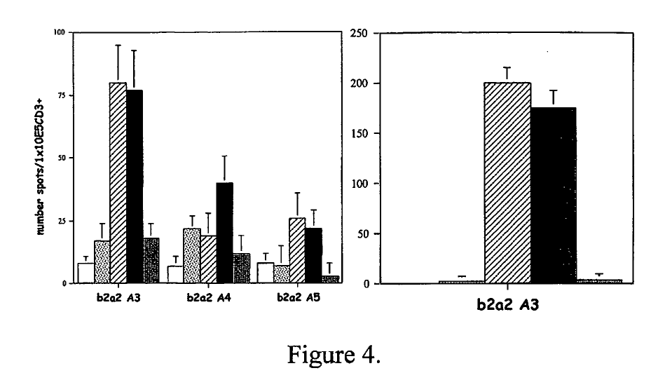

b2a2-derived peptides A3, A4 and A5 also generated a significant immune respose as measured by gamma-IFN secretion by CD3⁺ T cells (FIGS. 4A and 4B), with the response against A3 the most consistent between donors. A3-generated T cells recognized the native sequence as well, despite the fact that the native sequence is a weak HLA binder (Bocchia M, Wentworth Pa., et al, Blood. 1995; 85(10): 2680-4)

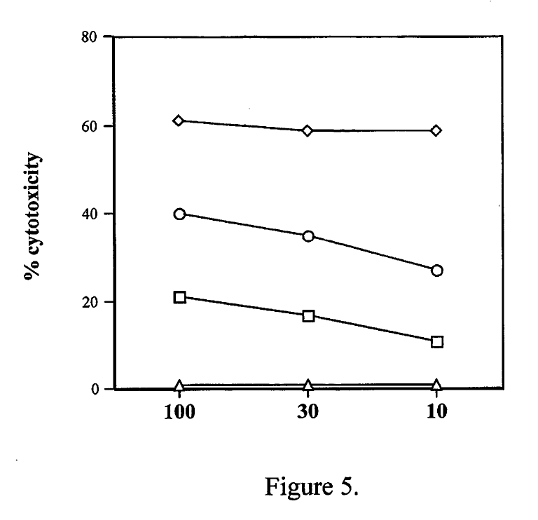

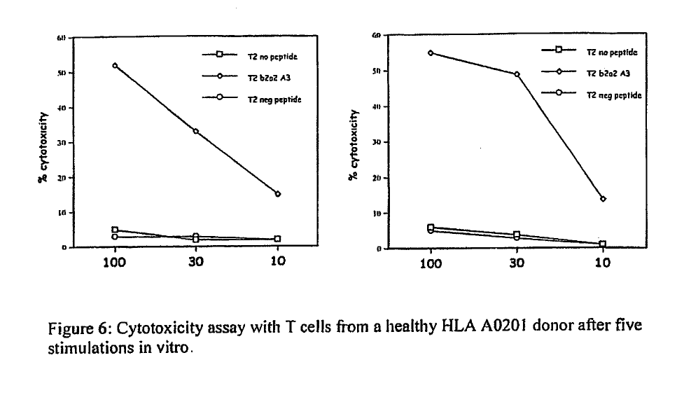

In order to determine whether the in vitro-generated T cells were capable of cytolysis, T cell lines obtained after several stimulations with p210C and b2a2A3 were assayed by chromium-51 release assays using peptide pulsed target cell lines. The cells were able to kill T2 cells pulsed with the heteroclitic peptides. In addition, the cells were able to recognize and kill cells expressing the native peptide from which the heteroclitic peptide was derived (FIGS. 5 and 6). As expected, the cells did not lyse T2 cells without peptide or T2 cells with control peptide, showing the specificity of the assay.

These results confirm the results of the previous Examples, showing that heteroclitic peptides of the present invention exhibit increased immunogenicity relative to the corresponding unmutated (“native”) sequences in both healthy and CML subjects. These results also show that T cells generated with the heteroclitic peptides can recognize MHC molecules bearing the native peptides, even when the native peptide is a weak binder, and can lyse target cells bearing the corresponding peptides. Thus, these results demonstrate the utility of heteroclitic peptides of the present invention in vaccinating subjects against bcr-abl-expressing cancer cells.

What is claimed is:

1. A bcr-abl-based peptide, comprising a sequence of amino acids that is an analogue peptide of a native bcr-abl breakpoint peptide that specifically binds to a HLA A0201 or HLA A0301 molecule on a cell surface,

2. The bcr-abl-based peptide of claim 1, wherein said bcr-abl-based peptide binds to said HLA A0201 or HLA A0301 molecule on a cell surface with a greater affinity than said native bcr-abl breakpoint peptide.

3. The bcr-abl-based peptide of claim 1, wherein said analogue peptide is a degradation product of said bcr-abl-based peptide.

4. The bcr-abl-based peptide of claim 1, wherein a total number of amino acids in said analogue peptide is about 70% to about 130% of a total number of amino acids in said native peptide.

5. The bcr-abl-based peptide of claim 1, wherein said analogue peptide has 8-12 amino acids.

6. The bcr-abl-based peptide of claim 1, wherein said native bcr-abl breakpoint peptide is a p210-b3a2 peptide, a p210-b2a2 peptide, or a p190-ela2 peptide.

7. The bcr-abl-based peptide of claim 1, said analogue peptide has an amino acid sequence selected from the group consisting of YLKALQRPV (SEQ ID NO: 2), KQSSKALQV (SEQ ID NO: 4), KLSSKALQV (SEQ ID NO: 5), KLLQRPVAV (SEQ ID NO: 7), TLFKQSSKV (SEQ ID NO: 9), YLFKQSSKV (SEQ I) NO: 10), LLINKEEAL (SEQ ID NO: 12), LTINKVEAL (SEQ ID NO: 13), YLINKEEAL (SEQ ID NO: 14), YLINKEEAV (SEQ ID NO: 15), and YLINKVEAL (SEQ ID NO: 16).

8. The bcr-abl-based peptide of claim 1, said native bcr-abl breakpoint peptide has an amino acid sequence selected from the group consisting of SSKALQRPV (SEQ ID NO: 1), KQSSKALQR (SEQ ID NO: 3), KALQRPVAS (SEQ ID NO: 6), TGFKQSSKA (SEQ ID NO: 8), or LTINKEEAL (SEQ ID NO: 11).

9. The bcr-abl-based peptide of claim 1, wherein said analogue peptide differs from said native bcr-abl breakpoint peptide in a major histocompatibility complex (MHC) molecule anchor residue.

10. An immunogenic composition comprising (a) the bcr-abl-based peptide of claim 1 or a DNA molecule encoding same; and (b) an adjuvant.

11. The immunogenic composition of claim 10, wherein said adjuvant is QS21, Freund's incomplete adjuvant, aluminum phosphate, aluminum hydroxide, BCG, or alum.

12. The immunogenic composition of claim 10, wherein said DNA is contained in a vector or an antigen presenting cell.

13. A vaccine comprising (a) the bcr-abl-based peptide of claim 1 or a DNA molecule encoding same and (b) a pharmaceutically acceptable carrier.

14. The vaccine of claim 13, wherein said DNA is contained in a vector or an antigen presenting cell.

15. The vaccine of claim 13, further comprising an immunogenic carrier associated therewith.

16. The vaccine of claim 15, wherein said immunogenic carrier is a protein, a peptide or an antigen-presenting cell.

17. The vaccine of claim 16, wherein said protein or peptide is keyhole limpet hemocyanin, an albumin or a polyamino acid.

18. The vaccine of claim 16, wherein said antigen presenting cell is a dendritic cell.

19. A method of inducing in a subject formation and proliferation of human cytotoxic T cells (CTL) that recognize a cancer cell, wherein said cancer cell presents the native bcr-abl breakpoint peptide of claim 1 on a major histocompatibility complex (MHC) class I molecule thereof, said method comprising contacting said subject with the bcr-abl-based peptide of claim 1, whereby said bcr-abl-based peptide induces formation and proliferation of said human CTL.

20. A method of treating a subject having a bcr-abl-expressing cancer, wherein a cell of said bcr-abl-expressing cancer presents the native bcr-abl breakpoint peptide of claim 19 on a major histocompatibility complex (MHC) class I molecule thereof, comprising
a. inducing in a donor formation and proliferation of human CTL that recognize said cell by the method of claim 19;
b. removing said human CTL from said donor; and
c. infusing said human CTL into said subject,
thereby treating a subject having a bcr-abl-expressing cancer.

21. The method of claim 19, whereby said bcr-abl-based peptide is produced by contacting said subject with a DNA molecule encoding same.

22. A method of treating a subject having a bcr-abl-expressing cancer, wherein a cell of said bcr-abl-expressing cancer presents the native bcr-abl breakpoint peptide of claim 1 on a major histocompatibility complex (MHC) class I molecule thereof, comprising
a. removing human immune cells from a donor;
b. contacting ex vivo said human immune cells with the bcr-abl-based peptide of claim 1, whereby said bcr-abl-based peptide induces formation and proliferation of cytotoxic T cells (CTL) that recognize said cell of said cancer;
c. infusing said CTL into said subject,
thereby treating a subject having a bcr-abl-expressing cancer.

23. A method of inducing a heteroclitic immune response against a bcr-abl-expressing leukemia cell in a human, the method comprising administering to said human an effective amount of a pharmaceutical composition, said pharmaceutical composition comprising:
a. a therapeutically effective amount of the bcr-abl-based peptide of claim 1 or a DNA encoding same; and
b. a pharmaceutically acceptable carrier,
whereby said bcr-abl-expressing leukemia cell presents the native bcr-abl breakpoint peptide of claim 1, thereby inducing formation and proliferation of cytotoxic T cells that recognize said bcr-abl-expressing leukemia cell.

24. The method of claim 23, wherein said human has an active cancer, is in remission from cancer or is at risk of developing a cancer.

25. A method for treating a bcr-abl-expressing leukemia in a subject, comprising inducing a heteroclitic immune response against a leukemia cell in a donor by the method of claim 23, and infusing the cytotoxic T-cells of claim 23 to said subject, thereby treating a bcr-abl-expressing leukemia in a subject.

26. The method of claim 25, wherein said leukemia is a chronic myelogenous leukemia.


SEQUENCE LISTING (18 sequences)

SEQ ID NO: 1 - protein, 9 aa, Homo sapiens
SSKALQRPV

SEQ ID NO: 2 - protein, 9 aa, Homo sapiens
YLKALQRPV

SEQ ID NO: 3 - protein, 9 aa, Homo sapiens
KQSSKALQR

SEQ ID NO: 4 - protein, 9 aa, Homo sapiens
KQSSKALQV

SEQ ID NO: 5 - protein, 9 aa, Homo sapiens
KLSSKALQV

SEQ ID NO: 6 - protein, 9 aa, Homo sapiens
KALQRPVAS

SEQ ID NO: 7 - protein, 9 aa, Homo sapiens
KLLQRPVAV

SEQ ID NO: 8 - protein, 9 aa, Homo sapiens
TGFKQSSKA

SEQ ID NO: 9 - protein, 9 aa, Homo sapiens
TLFKQSSKV

SEQ ID NO: 10 - protein, 9 aa, Homo sapiens
YLFKQSSKV

SEQ ID NO: 11 - protein, 9 aa, Homo sapiens
LTINKEEAL

SEQ ID NO: 12 - protein, 9 aa, Homo sapiens
LLINKEEAL

SEQ ID NO: 13 - protein, 9 aa, Homo sapiens
LTINKVEAL

SEQ ID NO: 14 - protein, 9 aa, Homo sapiens
YLINKEEAL

SEQ ID NO: 15 - protein, 9 aa, Homo sapiens
YLINKEEAV

SEQ ID NO: 16 - protein, 9 aa, Homo sapiens
YLINKVEAL

SEQ ID NO: 17 - protein, 9 aa, Artificial
  consensus motif
VYFFLPDHL

SEQ ID NO: 18 - protein, 9 aa, Influenza virus
GILGFVFTL